SEQUENCE LISTING (51 sequences)

SEQ ID NO: 1 - protein, 10 aa, synthetic construct
  REGION 1..10: note = cancer associated antigen
ELAGIGILTV

SEQ ID NO: 2 - protein, 23 aa, synthetic construct
  REGION 1..23: note = cancer associated antigen
RNGYRALMDKSLHVGTQCALTRR

SEQ ID NO: 3 - protein, 9 aa, synthetic construct
  REGION 1..9: note = example of a mutant APC antigen
QATEAERSF

SEQ ID NO: 4 - protein, 10 aa, synthetic construct
  REGION 1..10: note = cancer associated antigen
VVRHCPHHER

SEQ ID NO: 5 - protein, 10 aa, synthetic construct
  REGION 1..10: note = cancer associated antigen
EVVRHCPHHE

SEQ ID NO: 6 - protein, 9 aa, synthetic construct
  REGION 1..9: note = cancer associated antigen
AALLNKLYA

SEQ ID NO: 7 - protein, 9 aa, synthetic construct
  REGION 1..9: note = cancer associated antigen
EGSRNQDWL

SEQ ID NO: 8 - protein, 16 aa, synthetic construct
  REGION 1..16: note = cancer associated antigen
WNRQLYPEWTEAQRLD

SEQ ID NO: 9 - protein, 27 aa, synthetic construct
  REGION 1..27: note = cancer associated antigen
PSKPSFQEFVDWENVSPELNSTDQPFL

SEQ ID NO: 10 - protein, 27 aa, synthetic construct
  REGION 1..27: note = cancer associated antigen
EFKHIKAFDRTFANNPGPMVVFATPGM

SEQ ID NO: 11 - protein, 8 aa, synthetic construct
  REGION 1..8: note = OVA neoantigen
SIINFEKL

SEQ ID NO: 12 - protein, 17 aa, synthetic construct
  REGION 1..17: note = cancer associated antigen
ISQAVHAAHAEINEAGR

SEQ ID NO: 13 - protein, 15 aa, synthetic construct
  REGION 1..15: note = cancer associated antigen
GDFGLATEKSRWSGS

SEQ ID NO: 14 - protein, 9 aa, synthetic construct
  REGION 1..9: note = cancer associated antigen
YLSGADLNL

SEQ ID NO: 15 - protein, 9 aa, synthetic construct
  REGION 1..9: note = cancer associated antigen
ASMTNMELM

SEQ ID NO: 16 - protein, 28 aa, synthetic construct
  REGION 1..28: note = cancer associated antigen
GIPVHLELASMTNMELMSSIVHQQVFPT

SEQ ID NO: 17 - protein, 17 aa, synthetic construct
  REGION 1..17: note = cancer associated antigen
KLVVVGAGGVGKSALTI

SEQ ID NO: 18 - protein, 17 aa, synthetic construct
  REGION 1..17: note = cancer associated antigen
KLVVVGADGVGKSALTI

SEQ ID NO: 19 - protein, 15 aa, synthetic construct
  REGION 1..15: note = cancer associated antigen
SALTIQLIQNHFVDE

SEQ ID NO: 20 - protein, 15 aa, synthetic construct
  REGION 1..15: note = cancer associated antigen
FLCVFAINNTKSFED

SEQ ID NO: 21 - protein, 15 aa, synthetic construct
  REGION 1..15: note = cancer associated antigen
FYTLVREIRKHKEKM

SEQ ID NO: 22 - protein, 9 aa, synthetic construct
  REGION 1..9: note = Examples of BRCA mutated epitopes
YIHTHTFYV

SEQ ID NO: 23 - protein, 9 aa, synthetic construct
  REGION 1..9: note = Examples of BRCA mutated epitopes
SQIWNLNPV

SEQ ID NO: 24 - protein, 13 aa, synthetic construct
  REGION 1..13: note = An examples of a universal HLA-DR-binding T helper syntheticepitope
AKFVAAWTLKAAA

SEQ ID NO: 25 - protein, 7 aa, synthetic construct
  REGION 1..7: note = Secretion Signal Sequence
MISSSIS

SEQ ID NO: 26 - DNA, 24 bp, synthetic construct
  misc_feature 1..24: note = Secretion Signal Sequence
  source 1..24: mol_type = other DNA
atgatcagctctagttcaatcagc

SEQ ID NO: 27 - protein, 8 aa, synthetic construct
  REGION 1..8: note = Secretion Signal Sequence
MISSSSSI

SEQ ID NO: 28 - DNA, 24 bp, synthetic construct
  misc_feature 1..24: note = Secretion Signal Sequence
  source 1..24: mol_type = other DNA
atgatcagctctagttcaagcatc

SEQ ID NO: 29 - DNA, 66 bp, synthetic construct
  misc_feature 1..66: note = Secretion Signal Sequence
  source 1..66: mol_type = other DNA
atgaaatacctgttgcctacggcagccgctggattgttattactcgcggcccaaccggccatggcc

SEQ ID NO: 30 - protein, 11 aa, synthetic construct
ACDCRGDCFCG

SEQ ID NO: 31 - DNA, 172 bp, synthetic construct
  misc_feature 1..172: note = ompC promotor sequence
  source 1..172: mol_type = other DNA
ttttgaaacatctatgtagataactgtaacatcttaaaagttttagtatcatattcgtgttggattattctgtatttttgcggagaatggacttgccgactggttaatgagggttaaccagtaagcagtggcataaaaaagcaataaaggcatataacagagggttaataac

SEQ ID NO: 32 - DNA, 1521 bp, synthetic construct
  misc_feature 1..1521: note = salR pSal promotor sequence
  source 1..1521: mol_type = other DNA
ttacagcacgctaatcacttttttgcaggtctgaatcagccaaatgcgggccagcggttcgttggtctgggcaaaccaatgcaggcccacataaaattcgctcatctgaaacggcagttccagcacgcgcacatggctctggttcagatactgctgggcggcgcggctcggcagggtcaccatcagatcggttttatccagcacgccctgcagcacgccaaaatgcggcacttccagggcaattttgcgctgatggcccatcagtttcagcacatgatccacctgggtatggccggtgctgcttttaatcgccacatggctttcgttcagatacgcatcaaagctcagggtgctgcgaatgcgcggatggttcatgttcaccagggccacatagcgttccagaaacagggtttcatattccagatgtttaaattccagatgggtgctgttaaacacggccacatcaataaagccttcaatcagccatttttccacctgatcaattttaatttcttccacttcaattttaattttcggggcatgggcgcgcagatattcaatcagggtcggcagcaggcaaatttcgccaatatcgctcaggccaatgcgaaaggttttgttgctggtcagcgggttaaagttcagggcttcatccacggcaatttcaattttcagaatgctttctttaaacaccggatacagttcatggctcagtttggtggcttccacgccatactgggtgcgttcaaacagcgggttgttcagatgtttgcgcaggcggttcaggttataggtcacgctcggctggctcaggttcagaatttcggcggctttgctaatgtttttgttttcatacacgcaaataaaaatgcgaatcaggctcagatccatctagtatttctcctctttaatctctagtagctagcacagtccctaggactgagctagctgtaaaagtaaaatagctgcaatgagtttggctgatagtcgccagctgatcagttgtagaatgaaaaaataagtgcctgccagaatcaaaaaagttccagccaaaaatagatattgcatcaaaccttggaatgagctcaattcaatcagtcgttcataaaaacgagcatttgtaaaacttgctaaccaacaagctagaagcccattaaatgtatttatagataaacttacttttttgaattgtagcaatttaaacatctcaatatcgttcagttatttgaggggtatagctacattaaagagtatgaaaattaagagaaaattagttttcgcttcaatgattcaatttaaatctaaatatcaatgttttaagatcataatgatggatatgaaaacttaaacctataccggataggcaattaagaaagaaatatgatataattgttttaagatattgattttaataaattatttaaaaaattaataactgctttaaaaaactttaaattggatttaattgagtttgcagactaaaaataaaacaataaatcaagttctcaaaggaaatgagtc

SEQ ID NO: 33 - DNA, 35 bp, synthetic construct
  misc_feature 1..35: note = J23109 promotor sequence
  source 1..35: mol_type = other DNA
tttacagctagctcagtcctagggactgtgctagc

SEQ ID NO: 34 - DNA, 471 bp, synthetic construct
  misc_feature 1..471: note = mouse wt, Interleukin-18 (IL-18)
  source 1..471: mol_type = other DNA
aactttggccgccttcactgtacaaccgcagtaatacggaatataaatgaccaagttctcttcgttgacaaacgccagcctgtgttcgaggatatgactgatattgatcaaagtgccagtgaaccccagacccgcctgataatatacatgtacaaagacagtgaagtacgcggactggctgtgaccctctctgtgaaggatagtaaaatgtctaccctctcctgtaagaacaagatcatttcctttgaggaaatggatccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagt

SEQ ID NO: 35 - DNA, 471 bp, synthetic construct
  misc_feature 1..471: note = mouse CS2 mut Interleukin-18 (IL-18)
  source 1..471: mol_type = other DNA
cactttggccgccttcactgtacaaccgcagtaatacggaatataaatgaccaagttctcttcgttgacaaaagacagcctgtgttcgaggatatgactgatattgatcaaagtgccagtgaaccccagaccagactgataatatacgcgtacggtgacagtcgcgcaagaggaaaagctgtgaccctctctgtgaaggatagtaaaatgtctaccctctcctgtaagaacaagatcatttcctttgaggaaatggatccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagt

SEQ ID NO: 36 - DNA, 474 bp, synthetic construct
  misc_feature 1..474: note = human wt, Interleukin-18 (IL-18)
  source 1..474: mol_type = other DNA
tactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 37 - DNA, 471 bp, synthetic construct
  misc_feature 1..471: note = human 6-12 variant interleukin-18 (IL-18)
  source 1..471: mol_type = other DNA
tactttggcaagcttgaatctaaattatcagtcataagaaatttgaatgaccaagttctcttcattgaccaaggaaatcggcctctgtttgaagatatgactgattctgactgtagagataatgcaccccggaccatatttattataagtaagtatagcgatagcctggctagaggtctggctgtaactatctctgtgaagtgtgagaaaatttcaactctctcctgtgagaacaaaattatttcctttaaggaaatgaatcctcctgataacatcaaggatacaaaaagtgacatcatattctttcagagagatgtcccaggacatagccgcaagatgcaatttgaatcttcatcatacgaaggatactttctggcttgtgaaaaagagagagacctttttaaactcattttgaaaaaagaggatgaattgggggatagatctataatgttcactgttcaaaacgaagac

SEQ ID NO: 38 - DNA, 720 bp, synthetic construct
  misc_feature 1..720: note = Necrosis Factor Superfamily Member 14 (LIGHT) (adapted for human and mouse Tumor)
  source 1..720: mol_type = other DNA
atggaagaatctgtcgtaagaccgtcggtatttgttgtagatggacaaacagatattccgtttacgcgtttaggtcgttcacatcgtcggcaatcatgttcagtcgctcgtgtcgggttaggacttttgttgttattaatgggtgcgggtttggcagttcaagggtggtttctcttacaacttcattggcggcttggtgaaatggttacgcgtttgccagatggaccggctggttcctgggaacaattgattcaagaacgacgttcccatgaagttaatccggcggcacatttaacaggtgcaaattcgagtcttactggatcgggtggacctttgctttgggaaacacaattggggttagcatttctccggggtttgtcctatcatgatggggcgttagttgtaacgaaaacgggttattattatatatattcaaaagtccaacttggcggtgttgggtgtccgctcggattagcaggtactattactcatggattatataaaagaacaccacggtatcctgaagaattggaattattagtttcgcaacaaagtccatgtggacgggctacgtcgtcatccagagtttggtgggatagttcgttcttgggtggggtcgtccatcttgaagcaggggagaaagtcgtagttcgtgttttaggtaaacgtttagtcagattacgagatggaacacggagttattttggggcatttatggtc

SEQ ID NO: 39 - DNA, 720 bp, synthetic construct
  misc_feature 1..720: note = human wt, Tumor Necrosis Factor Superfamily Member 14 (LIGHT)
  source 1..720: mol_type = other DNA
atggaggagagtgtcgtacggccctcagtgtttgtggtggatggacagaccgacatcccattcacgcgcctgggacgcagccaccggcgccagtcgtgcagtgtggcccgggtgggtctgggtctcttgctgttgctgatgggggctgggctggccgtccaaggctggttcctcctgcagctgcactggcgtctgggagagatggtcacccgcctgcctgacggacctgcaggctcctgggagcagctgatacaagagcgccgctctcacgaggtcaacccagcagcgcatctcacaggggccaactccagcttgaccggcagcggggggccgctgttatgggagactcagctgggcctggccttcctgcgcggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagaaggtggtcgtccgtgtgctggatgaacgcctggttcgcctgcgtgatggtacccggtcttacttcggggctttcatggtg

SEQ ID NO: 40 - DNA, 816 bp, synthetic construct
  misc_feature 1..816: note = human hCD40-Tri, CD40 Ligand (CD40L)
  source 1..816: mol_type = other DNA
caccatcaccatcaccatcaccatggcgaccgcatgaagcagatcgaggacaagatcgaggagatcctgagcaagatctaccacatcgagaacgagatcgcccgcatcaagaagctgatcggcgagcgcaccagcggcggtagcggaggcaccggtggaagcggcggtaccggaggcagcgacaagatcgaggacgagcgcaacctgcacgaggacttcgtgttcatgaagaccatccagcgctgcaacaccggcgagcgcagcctgagcctgctgaactgcgaggagatcaagagccagttcgagggcttcgtgaaggacatcatgctgaacaaggaggagaccaagaaggagaacagcttcgagatgcagaagggcgaccagaacccccagatcgccgcccacgtgatcagcgaggccagcagcaagaccaccagcgtgctgcagtgggccgagaagggctactacaccatgagcaacaacctggtgaccctggagaacggcaagcagctgaccgtgaagcgccagggcctgtactacatctacgcccaggtgaccttctgcagcaaccgcgaggccagcagccaggcccccttcatcgccagcctgtgcctgaagagccccggccgcttcgagcgcatcctgctgcgcgccgccaacacccacagcagcgccaagccctgcggccagcagagcatccacctgggcggcgtgttcgagctgcagcccggcgccagcgtgttcgtgaacgtgaccgaccccagccaggtgagccacggcaccggcttcaccagcttcggcctgctgaagctgtaa

SEQ ID NO: 41 - DNA, 813 bp, synthetic construct
  misc_feature 1..813: note = mouse mCD40-Tri, CD40 Ligand (CD40L)
  source 1..813: mol_type = other DNA
caccatcaccatcaccatcaccatggcgaccgcatgaagcagatcgaggacaagatcgaggagatcctgagcaagatctaccacatcgagaacgagatcgcccgcatcaagaagctgatcggcgagcgcaccagcggcggtagcggaggcaccggtggaagcggcggtaccggaggcagcgacaaggtggaggaggaggtgaacctgcacgaggacttcgtgttcatcaagaagctgaagcgctgcaacaagggcgagggcagcctgagcctgctgaactgcgaggagatgcgccgccagttcgaggacctggtgaaggacatcaccctgaacaaggaggagaagaaggagaacagcttcgagatgcagcgcggcgacgaggacccccagatcgccgcccacgtggtgagcgaggccaacagcaacgccgccagcgtgctgcagtgggccaagaagggctactacaccatgaagagcaacctggtgatgctggagaacggcaagcagctgaccgtgaagcgcgagggcctgtactacgtgtacacccaggtgaccttctgcagcaaccgcgagcccagcagccagcgccccttcatcgtgggcctgtggctgaagcccagcagcggcagcgagcgcatcctgctgaaggccgccaacacccacagcagcagccagctgtgcgagcagcagagcgtgcacctgggcggcgtgttcgagctgcaggccggcgccagcgtgttcgtgaacgtgaccgaggccagccaggtgatccaccgcgtgggcttcagcagcttcggcctgctgaagctgtaa

SEQ ID NO: 42 - DNA, 1146 bp, synthetic construct
  misc_feature 1..1146: note = human ompC-hCD40p-ompC CD40 Ligand (CD40L)
  source 1..1146: mol_type = other DNA
atgaaagttaaagtactgtccctcctggtaccagctctgctggtggcgggcgcagcgaatgcggctgaaatttataataaagacggcaacaaattagacctgtttggtaaagttgatggcctgcactacttctctgacgacaaaggcagcgatggcgaccagacctacatgcgtatcggcttcaaaggcgaaacgcaggttaacgatcagctgaccggttatggccagtgggaatatcagattcagggcaaccagactgaaggcagcaacgactcctggacgcgtgtggcgtttgcgggtctgaaattcgctgatgcaggttccttcgattatggtcgtaactacggcgtaacctatgacgtgacctcctggaccgacgttctgccggagttcggcggcgacacctacggcgctgacaactttatgcagcagcgtggtaacggctatgctacctaccgtaacaccgacttcttcggcctggtggatggtctggacttcgcgttacagtatcagggcaaaaacggcagcgtgagcggtgaaaacaccaacggtcgcagcctgctgaaccagaacggcgacggttacggcggatcgctgacttatgcaatcggcgaaggcttctctgtcggtggcgctatcaccacgtctaaacgtactgccgatcagtgggctgaaaaaggttactacactatgtccctgtatggtaacggcgatcgcgccacggtttacaccggcggcctgaaatacgatgcgaacaacatctatctggcagcgcagtattctcagacctataacgcaacccgttttggtacctctaacggtagcaacccgtccacctcttacggttttgccaacaaagcgcagaactttgaagtggttgctcagtaccagttcgactttggtctgcgtccgtctgtggcttacctgcagtctaaaggtaaggacatcagcaacggttacggcgccagctatggcgaccaggacatcgtaaaatacgttgatgtcggcgcgacttactacttcaacaaaaacatgtccacctatgttgattacaaaatcaacctgctggataaaaacgactttacccgcgatgcgggcatcaacaccgacgacatcgtagcgctgggtctggtttaccagttctaa

SEQ ID NO: 43 - DNA, 1149 bp, synthetic construct
  misc_feature 1..1149: note = mouse ompC-mCD40p-ompC CD40 Ligand (CD40L)
  source 1..1149: mol_type = other DNA
atgaaagttaaagtactgtccctcctggtaccagctctgctggtggcgggcgcagcgaatgcggctgaaatttataataaagacggcaacaaattagacctgtttggtaaagttgatggcctgcactacttctctgacgacaaaggcagcgatggcgaccagacctacatgcgtatcggcttcaaaggcgaaacgcaggttaacgatcagctgaccggttatggccagtgggaatatcagattcagggcaaccagactgaaggcagcaacgactcctggacgcgtgtggcgtttgcgggtctgaaattcgctgatgcaggttccttcgattatggtcgtaactacggcgtaacctatgacgtgacctcctggaccgacgttctgccggagttcggcggcgacacctacggcgctgacaactttatgcagcagcgtggtaacggctatgctacctaccgtaacaccgacttcttcggcctggtggatggtctggacttcgcgttacagtatcagggcaaaaacggcagcgtgagcggtgaaaacaccaacggtcgcagcctgctgaaccagaacggcgacggttacggcggatcgctgacttatgcaatcggcgaaggcttctctgtcggtggcgctatcaccacgtctaaacgtactgccgatcagtgggccaagaagggctactacaccatgaagagcctgtatggtaacggcgatcgcgccacggtttacaccggcggcctgaaatacgatgcgaacaacatctatctggcagcgcagtattctcagacctataacgcaacccgttttggtacctctaacggtagcaacccgtccacctcttacggttttgccaacaaagcgcagaactttgaagtggttgctcagtaccagttcgactttggtctgcgtccgtctgtggcttacctgcagtctaaaggtaaggacatcagcaacggttacggcgccagctatggcgaccaggacatcgtaaaatacgttgatgtcggcgcgacttactacttcaacaaaaacatgtccacctatgttgattacaaaatcaacctgctggataaaaacgactttacccgcgatgcgggcatcaacaccgacgacatcgtagcgctgggtctggtttaccagttctaa

SEQ ID NO: 44 - DNA, 360 bp, synthetic construct
  misc_feature 1..360: note = NOD_SE variant, Signal Regulatory Protein Alpha (SIRPa) (adapted for human and mouse)
  source 1..360: mol_type = other DNA
gcaacgcggacagaagtcaaagtcattcaaccggaaaagtcagtttccgtagcagctggtgattccacggtattgaattgtacgttgacatcacttcttcctgttgggccgattcggtggtatagaggtgttggtcaatcgcggcagttaatatattcctttacaactgaacatttcccacgtgtcacgaatgtttctgatgctacgaagagatccaatttagacttcagtatcagaatttccaatgttacgcctgaagacgcgggaacttactattgtgttaaatttcagagaggttcaagtgaaccggatacagaaatacaatctggcggtggaacggaagtctatgttcttgcgaaa

SEQ ID NO: 45 - DNA, 207 bp, synthetic construct
  misc_feature 1..207: note = C-C Motif Chemokine Ligand 5 (CCL5)
  source 1..207: mol_type = other DNA
tgcccatattcctgcgacaccacaccctgctgctttgcctacattgcccgcccactgccccgtgcccacatcaaggagtatttctacaccagtggcaagtgctccaacccagcagtcgtctttgtcacccgcaagaaccgccaagtgtgtgccaacccagagaagaaatgggttcgggagtacatcaactctttggagatgagctaa

SEQ ID NO: 46 - DNA, 36 bp, synthetic construct
  misc_feature 1..36: note = human Anti-IL10R1 peptide
  source 1..36: mol_type = other DNA
ttccgcagcttcgagagctgcctggccaagagccac

SEQ ID NO: 47 - DNA, 432 bp, synthetic construct
  misc_feature 1..432: note = Granulocyte-macrophage colony stimulating factor (GM-CSF) (human (cross reactivity with mouse))
  source 1..432: mol_type = other DNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctgcagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggag

SEQ ID NO: 48 - DNA, 405 bp, synthetic construct
  misc_feature 1..405: note = human C-C Motif Chemokine Ligand 21 (CCL21)
  source 1..405: mol_type = other DNA
atggctcagtcactggctctgagcctccttatcctggttctggcctttggcatcccccgcacccaaggcagtgatggaggggctcaggactgttgcctcaagtacagccaacgcaagattcccgccaaggttgtccgcagctaccggaagcaggaaccaagcttaggctgctccatcccagctatcctgttcttgccccgcaagcgctctcaggcagagctgtgtgcagacccaaaggagctctgggtgcagcagctgatgcagcatctggacaagacaccatccccacagaaaccagcccagggctgccgcaaggaccgcggggcctccaagactggcaagaaaggaaagggctccaaaggctgcaagcgcactgagcggtcacagacccctaaagggccataa

SEQ ID NO: 49 - DNA, 399 bp, synthetic construct
  misc_feature 1..399: note = mouse C-C Motif Chemokine Ligand 21 (CCL21)
  source 1..399: mol_type = other DNA
atggctcagatgatgactctgagcctccttagcctggtcctggctctctgcatcccctggacccaaggcagtgatggagggggtcaggactgctgccttaagtacagccagaagaaaattccctacagtattgtccgcggctatcgcaagcaagaaccaagtttaggctgtcccatcccggcaatcctgttctcaccccggaagcactctaagcctgagctgtgtgcaaaccctgaggaaggctgggtgcagaacctgatgcgccgcctggaccagcctccagccccagggaaacaaagccccggctgccgcaagaaccggggaacctctaagtctggaaagaaaggaaagggctccaagggctgcaagagaactgaacagacacagccctcaagagga

SEQ ID NO: 50 - DNA, 735 bp, synthetic construct
  misc_feature 1..735: note = Short salmonella flagellin B (fliC)
  source 1..735: mol_type = other DNA
atggacgacgccgccggccaggccatcgccaacaggttcaccgccaacatcaagggcctgacccaggccagcaggaacgccaacgacggcatcagcatcgcccagaccaccgagggcgccctgaacgagatcaacaacaacctgcagagggtgagggagctggccgtgcagagcgccaacagcaccaacagccagagcgacctggacagcatccaggccgagatcacccagaggctgaacgagatcgacagggtgagcggccagacccagttcaacggcgtgaaggtgctggcccaggacaacaccctgaccatccaggtgggcgccaacgacggcgagaccatcgacatcgacctgaagcagatcaacagccagaccctgggcctggacaccctgaacgtgcagatcagcggcggcggcggcggcatcctggacagcatgggcgaggccgccgccaccaccaccgagaaccccctgcagaagatcgacgccgccctggcccaggtggacaccctgaggagcgacctgggcgccgtgcagaacaggttcaacagcgccatcaccaacctgggcaacaccgtgaacaacctgaccagcgccaggagcaggatcgaggacagcgactacgccaccgaggtgagcaacatgagcagggcccagatcctgcagcaggccggcaccagcgtgctggcccaggccaaccaggtgccccagaacgtgctgagcctgctgagg

SEQ ID NO: 51 - DNA, 522 bp, synthetic construct
  misc_feature 1..522: note = DacA
  source 1..522: mol_type = other DNA
atgtatggatcacgcattgagcgtgaacaacaccaccttattgagtctattgaaaagtccacgcaatacatggcgaagcgtcgcattggagctttgatctctgtggctcgtgatacaggcatggacgactacatcgagactggcattccgcttaatgcgaaaatctcatcgcaattattgattaatatcttcatccccaatacccctcttcacgatggcgcagtgatcattaaaggtaacgagatcgcgagcgctgccagttatctgccattgtccgactcgccgtttctttctaaggagctgggaactcgccatcgtgccgcattagggatttccgaggtgacagattcaatcacaatcgttgtcagcgaggaaacaggtgggatttcccttacgaagggaggcgaactgttccgtgatgtatccgaagaagaattgcataagattttgcttaaagagctggtgactgtcacagctaaaaaaccaagcattttctccaaatggaaaggaggtaagtccgag

(19) United States
(12) Patent Application Publication
(10) Pub. No.: US 2026/0048107 A1
(43) Pub. Date: Feb. 19, 2026

(54) GENETICALLY MODIFIED BACTERIA FOR GENERATING VACCINES

(71) Applicants: Yeda Research and Development Co. Ltd., Rehovot (IL); Baccine Ltd., Rehovot (IL)

(72) Inventors: Ravid Straussman, Mazkeret Batia (IL); Oded Sandler, Rehovot (IL); Reut Riff, Rehovot (IL); Debora Gitta Rosenberg, Rehovot (IL)

(73) Assignees: Yeda Research and Development Co. Ltd., Rehovot (IL); Baccine Ltd., Rehovot (IL)

(21) Appl. No.: 19/103,676

(22) PCT Filed: Aug. 17, 2023
(86) PCT No.: PCT/IL2023/050876

§ 371 Date: Feb. 13, 2025

(30) Foreign Application Priority Data
Aug. 17, 2022 (IL) 295726

(51) Int. Cl. A61K 39/00; A61P 35/00; C07K 14/54; C07K 14/77; C12N 1/20; C12N 9/12; C12R 1/42

(52) U.S. Cl. CPC A61K 39/00114; A61K 39/001162; A61P 35/00; C07K 14/54; C07K 14/77; C12N 1/20; C12N 9/1205; A61K 2039/522; A61K 2039/523; A61K 2039/54; A61K 2039/545; C07K 2319/02; C12R 2001/42

(57) ABSTRACT

A vaccine is disclosed which comprises a pharmaceutically acceptable carrier and Gramnegative bacteria, genetically modified to express: at least one disease-associated antigen, linked to a signal sequence belonging to a type II secretion system; and the at least one disease-associated antigen linked to a signal sequence belonging to a type III secretion system. Uses thereof are also disclosed.

### RELATED APPLICATIONS

This application claims the benefit of priority Israel Patent Application No. 295726 filed 17 Aug. 2022, the contents of which are incorporated herein by reference in their entirety.

### SEQUENCE LISTING STATEMENT

The XML file, entitled 97171 Sequence Listing.xml, created on 17 Aug. 2023, comprising 75,322 bytes, submitted concurrently with the filing of this application is incorporated herein by reference.

### FIELD AND BACKGROUND OF THE INVENTION

The present invention, in some embodiments thereof, relates to bacterial vaccines which may be manipulated to produce disease-associated antigens.

Advances in the understanding of molecular biology, the ability to predict immunogenic neoantigens by next generation sequencing and prediction algorithms, the lifestyles of pathogenic bacteria, bacterial engineering and synthetic biology tools have significantly accelerated the rational design of bacteria as antigen delivery vectors. Being a strong immunogen, bacteria may trigger a vast immune response against itself and consequently against the delivered neoantigen. Indeed, bacterial vectors that deliver antigenic messages are also able to deliver a strong danger signal mediated by their pathogen-associated molecular patterns (PAMPs), such as lipopolysaccharides, lipoproteins, flagellin and CpG. PAMPs derived from different classes of pathogens bind to diverse families of pathogen recognition receptors (PRRs) that include Toll-like receptors (TLRs), C-type lectin-like receptors (CLRs), retinoic acid-inducible gene (RIG)-like receptors (RLRs) and nucleotide-binding oligomerization domain (NOD)-like receptors (NLRs). These interactions according to each pathogen trigger distinct signaling pathways to differentially activate antigen presenting cells (APCs), thereby directing the adaptive effector response in a manner that is specifically adapted to the microbe and hence to the antigen delivered by the bacteria. Moreover, specialized toxins that bacteria use for their own virulence can reinforce effector or memory responses.

Background art includes U.S. patents application Nos. 20200087703, 20200054739 and 20190365830, Gopalakrishnan V et al, Science. 2018 Jan. 5; 359 (6371): 97-103; Geller et al., Science, Vol 357, Issue 6356 15 Sep. 2017; Riquelme E et al Cell. 2019 Aug. 8; 178 (4): 795-806.e12. doi: 10.1016/j.cell.2019.07.008; Straussman R et al., Nature. 2012 Jul. 26; 487 (7408): 500-4. doi: 10.1038/nature11183.

### SUMMARY OF THE INVENTION

According to an aspect of the present invention there is provided an attenuated bacteria of the species Salmonella enterica genetically modified to express a reduced amount or a less active product of at least one gene selected from the group consisting of arginine deaminase (adI), L-asparaaginase II (asnB), Aminoglycoside (3″) (9) adenylyltransferase (aadA), AAC (6′)-Iaa (aac6) and Tetrathionate reductase A (ttrA) as compared to non-attenuated bacteria of the species Salmonella enterica.

According to embodiments of the present invention, the attenuated bacteria is of a serotype Typhimurium.

According to embodiments of the present invention, the attenuated bacteria are of a strain STM3120.

According to embodiments of the present invention, the attenuated bacteria are capable of homing to a tumor of a subject having cancer following i.v. administration.

According to an aspect of the present invention there is provided a vaccine comprising the attenuated bacteria described herein, genetically modified to express at least one disease-associated antigen and a pharmaceutically acceptable carrier.

According to embodiments of the present invention, the bacteria are genetically modified to express at least one cancer-associated antigen.

According to embodiments of the present invention, the at least one cancer-associated antigen comprises a signal sequence.

According to embodiments of the present invention, the bacteria are genetically modified to express at least two cancer-associated antigens, wherein a first of the at least two cancer-associated antigens comprises a first signal sequence and a second of the at least two cancer-associated antigens comprises a second signal sequence, the first signal sequence belonging to a type II secretion system and the second signal sequence belonging to a type III secretion system.

According to embodiments of the present invention, the bacteria are genetically modified to express at least three cancer-associated antigens, wherein a first of the at least three cancer-associated antigens comprises a first signal sequence, a second of the at least three cancer-associated antigens comprises a second signal sequence, and a third of the at least three cancer-associated antigens comprises a sequence for embedding into an outer wall of the bacteria, the first signal sequence belonging to a type II secretion system and the second signal sequence belonging to a type III secretion system.

According to embodiments of the present invention, the bacteria are genetically modified to express an immunomodulator.

According to embodiments of the present invention, the immunomodulator is selected from the group consisting of Interleukin-18 (IL-18), Tumor Necrosis Factor Superfamily Member 14 (LIGHT), Signal Regulatory Protein Alpha (SIRPa), CD40 Ligand (CD40L), C—C Motif Chemokine Ligand 5 (CCL5), Anti-IL10R1 peptide, Granulocyte-macrophage colony stimulating factor (GM-CSF), C—C Motif Chemokine Ligand 21 (CCL21), Short Salmonella flagellin B (fliC) and DacA.

According to embodiments of the present invention, the immunomodulator is expressed in the bacteria under the control of an inducible promoter.

According to embodiments of the present invention, the inducible promoter is an aspirin inducible promoter.

According to an aspect of the present invention there is provided vaccine comprising a ghost bacteria of the species Salmonella enterica, the bacteria being genetically modified to express at least one disease-associated antigen on a cell wall of the bacteria.

According to embodiments of the present invention, the at least one disease-associated antigen is a cancer-associated antigen.

According to embodiments of the present invention, the bacteria are genetically modified to impair survival of bacteria in macrophages and reduce resistance to antibiotic without substantially affecting bacterial fitness and capability for tumor colonization.

According to embodiments of the present invention, the bacteria are genetically modified to express a reduced amount or an inactive product of a gene selected from the group consisting of arginine deaminase (adI), L-asparaaginase II (asnB), Aminoglycoside (3″) (9) adenylyltransferase (aadA), AAC (6′)-Iaa (aac6) and Tetrathionate reductase A (ttrA).

According to embodiments of the present invention, the bacteria are of a serotype Typhimurium.

According to embodiments of the present invention, the bacteria are of a strain STM3120.

According to embodiments of the present invention, the bacteria are capable of homing to a tumor of a subject having cancer following i.v. administration.

According to an aspect of the present invention there is provided method of treating cancer of a subject in need thereof comprising administering to the subject a therapeutically effective amount of the vaccine described herein, thereby treating the cancer.

According to embodiments of the present invention, the method further comprises administering to the subject:
(i) the vaccine described herein; and
(ii) a second vaccine comprising viable bacteria which is genetically modified to express at least one cancer-associated antigen, thereby treating the cancer.

According to embodiments of the present invention, the second vaccine is administered following the vaccine described herein.

According to embodiments of the present invention, the second vaccine is administered concomitantly with said vaccine described herein.

According to embodiments of the present invention, the cancer is selected from the group consisting of breast, melanoma, colorectal cancer, lung cancer, gastric cancer, pancreatic cancer, ovarian cancer, bone cancer and brain cancer.

According to embodiments of the present invention, the brain cancer comprises glioblastoma.

According to an aspect of the present invention there is provided a method of preventing cancer of a subject in need thereof the method comprising administering to the subject a prophylactically effective amount of the vaccine described herein, thereby preventing the cancer.

According to embodiments of the present invention, the cancer is selected from the group consisting of breast, melanoma, colorectal cancer, gastric cancer, lung cancer, pancreatic cancer, ovarian cancer, bone cancer and brain cancer.

According to embodiments of the present invention, the brain cancer comprises glioblastoma.

According to another aspect of the invention there is provided a vaccine comprising a pharmaceutically acceptable carrier and Gram-negative bacteria, genetically modified to express:
at least one disease-associated antigen, linked to a signal sequence belonging to a type II secretion system; and
the at least one disease-associated antigen linked to a signal sequence belonging to a type III secretion system.

According to embodiments of the invention, the bacteria are genetically modified to further express:
at least one disease-associated antigen linked to a bacterial outer membrane targeting sequence.

According to embodiments of the invention, the Gram-negative bacteria comprise a bacterium which is genetically modified to co-express:
the at least one disease-associated antigen, linked to the signal sequence belonging to a type II secretion system; and
the at least one disease-associated antigen linked to the signal sequence belonging to a type III secretion system.

According to embodiments of the invention, the Gram negative bacteria are of the species Salmonella enterica.

According to embodiments of the invention, the cancer-associated antigen is expressed in the bacteria under control of a constitutive promoter.

According to embodiments of the invention, the bacteria are genetically modified to express a reduced amount or an inactive product of a gene selected from the group consisting of: STM3120, arginine deaminase (adI), L-asparaaginase II (asnB), Aminoglycoside (3″) (9) adenylyltransferase (aadA), AAC (6′)-Iaa (aac6) and Tetrathionate reductase A (ttrA).

According to embodiments of the invention, the bacteria are genetically modified to express a reduced amount or an inactive product of three genes selected from the group consisting of STM3120, arginine deaminase (adI), L-asparaaginase II (asnB), Aminoglycoside (3″) (9) adenylyltransferase (aadA), AAC (6′)-Iaa (aac6) and Tetrathionate reductase A (ttrA).

According to embodiments of the invention, the bacteria are of a serotype Typhimurium.

According to embodiments of the invention, at least one of the three genes is STM3120.

According to embodiments of the invention, the bacteria are capable of homing to a tumor of a subject having cancer following i.v. administration.

According to an aspect of the invention, there is provided attenuated bacteria of the species Salmonella enterica genetically modified to express a reduced amount or a less active product of at least one gene selected from the group consisting of arginine deaminase (adI), L-asparaaginase II (asnB), Aminoglycoside (3″) (9) adenylyltransferase (aadA), AAC (6′)-Iaa (aac6) and Tetrathionate reductase A (ttrA) as compared to non-attenuated bacteria of the species Salmonella enterica.

According to embodiments of the invention, the bacteria further comprise a mutation in STM3120.

According to embodiments of the invention, the attenuated bacteria are capable of homing to a tumor of a subject having cancer following i.v. administration.

According to embodiments of the invention, the at least one cancer-associated antigen comprises a signal sequence.

According to embodiments of the invention, the bacteria are genetically modified to express at least two cancer-associated antigens, wherein a first of the at least two cancer-associated antigens comprises a first signal sequence and a second of the at least two cancer-associated antigens comprises a second signal sequence, the first signal sequence belonging to a type II secretion system and said second signal sequence belonging to a type III secretion system.

According to embodiments of the invention, the bacteria are genetically modified to express at least three cancer-associated antigens, wherein a first of the at least three cancer-associated antigens comprises a first signal sequence, a second of the at least three cancer-associated antigens comprises a second signal sequence, and a third of the at least three cancer-associated antigens comprises a sequence for embedding into an outer wall of the bacteria, the first signal sequence belonging to a type II secretion system and the second signal sequence belonging to a type III secretion system.

According to embodiments of the invention, the immunomodulator is expressed in the bacteria under control of an inducible promoter.

According to embodiments of the invention, the administering is by intravenous (i.v.) injection.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B. Long-term efficacy of vaccination by OVA expressing bacteria in B16-OVA tumor model. (A) Experiment timeline. (B) Tumor growth curves from start of treatment.

FIGS. 2A-G: Short-term efficacy and immunogenicity of vaccination by OVA expressing bacteria in B16-OVA tumor model. (A) Experiment timeline. (B) Tumor growth curves from start of treatment. N=5 for all mice cohorts. (C) Tumor volume percentage at day 16 relative to day 0. P-values were obtained by two sided Mann-Whitney test. Below are the percentage values of fully cured mice per cohort. (D) Mice from the cohort treated with Anti-PD1 or Anti-PD1 together with PACMAN-OVA. While the tumor of the mouse treated with PACMAN-OVA gradually disappears, the tumor of the mouse treated with anti-PD1 only continued to grow exponentially. (E) CFU count of tumor and liver extracts. Following 16 days from vaccination, bacteria from tumors and livers were seeded on LB plates with resistance to AMP. Per mouse, CFU count, and tumor volumes are given. Of note, 4 out of 5 mice of the PACMAN-OVA cohort exhibited complete clearance of bacteria. Bacteria were present in the mouse with the biggest tumor, suggesting that the tumor tissue enables bacteria proliferation. (F) Sera of mice cohorts were subjected to IFNg ELISA. The PACMAN-OVA cohort exhibited the highest IFNg serum level indicating high systemic immune activation. Green dot refers to mouse 836 in F. Mouse 836 was the only case where bacteria were present in the liver, probably resulting in the highest serum level of IFNg. (G) Quantification of SIINFEKL (SEQ ID NO: 11) specific TCR by Flow Cytometry. To quantify neoantigen specific T cell clones, splenocyte test were co-incubated with Tetramer presenting the OVA neoantigen (SIINFEKL—SEQ ID NO: 11)) and conjugated to a fluorescent dye. Thus, splenocytes which are positive to the Tetramer dye possess a TCR that can bind the OVA neoantigen. Following FACS, percentage of SIINFEKEL (SEQ ID NO: 11) positive T cells out of CD3+CD8+ population was the highest among mice vaccinated with the PACMAN-OVA. P-values were obtained by Two sided Mann-Whitney test.

FIGS. 3A-D. Alternate administration of different bacterial vaccines may overcome acquired immunity. (A) Experiment timetable. (B) Tumor growth curves. (C) Weight change (percentage of initial weight) during the first 24 days of treatment. Mice cohorts are: STM3120+aPD1 (N=3), STM-pagC-SspH1-OVA+aPD1 (N=3), STM-SspH2-OVA+aPD1 (N=5). Dashed lines demarcate day of bacteria administration. (D) Weight change (percentage of initial weight) during the first 40 days of treatment. Mice cohorts are: STM3120+aPD1 (N=3), STM-pagC-SspH1-OVA+aPD1 (N=3), STM-SspH2-OVA+aPD1 (N=5), CHA-OST. Dashed lines demarcate day of bacteria administration.

FIGS. 4A-B. Immune memory of vaccination by OVA expressing bacteria in B16-OVA tumor model. (A) Experiment timetable. (B) Tumor growth curves.

FIGS. 5A-C. Long-term efficacy of vaccination by Adpgk expressing bacteria in MC38 CRC tumor model. (A) Experiment timeline. (B) Tumor growth curves from treatment start for anti-PD1 (75 μg per mouse, i.p, once a week), mice receiving anti-PD1 together with VNP20009 and mice receiving anti-PD1 together with PACMAN-Adpgk (10⁶ CFU, tail vein). (C) Tumor growth curves for re-challeng where 10⁵ MC38 cells were reintroduced and tumor growth was compared to naïve mice injected with the same number of cells.

FIG. 6 is a graph illustrating tumor colonizing of attenuated (STM3120) Salmonella bacteria.

FIG. 7 is a graph illustrating toxicity of i.v. administration of attenuated (STM3120) vs parental (14028) Salmonella.

FIGS. 8A-B are graphs illustrating splenocytes immune profiling following vaccination by OVA expressing bacteria in B16-OVA tumor model. FIG. 8A—Quantification of IFNg positive CD8 T-cells by FACS. FIG. 8B—Quantification of T cells killing capacity.

FIGS. 9A-C illustrate long-term efficacy of vaccination by Adpgk expressing P. aeruginosa in MC38 tumor model. FIG. 9A—Experiment timetable. FIG. 9B—Tumor growth curves. Mice treated with PACMAN-ADPGK i.v, exhibited a considerable delayed tumor growth. Per treatment, indicated number of fully cured mice. FIG. 9C—Average tumor growth curves of the mice cohorts in FIG. 9B. Whiskers indicate standard error.

FIGS. 10A-B illustrates long-term efficacy of vaccination by Adpgk expressing Bacillus Subtilis spores in MC38 tumor model. FIG. 10A—Experimental timetable. FIG. 10B. Tumor growth curves. Mice treated with PACMAN-ADPGK spores p.o or i.v, exhibited a considerable delayed tumor growth. Per treatment, indicated number of fully cured mice.

FIGS. 11A-B illustrates long-term efficacy of vaccination by Adpgk expressing attenuated Salmonella (STM3120) in MC38 tumor model. FIG. 11A. Experiment timetable. FIG. 11B—Tumor growth curves. Mice treated with PACMAN-ADPGK exhibited a considerable delayed tumor growth. Per treatment, indicated number of fully cured mice.

FIGS. 12A-B. The effect of knockout of Salmonella virulence genes on systemic toxicity and tumor homing. Mice bearing MC38 sub-cutaneous tumors (>100 mm\hat{ }3) were injected I.V with the Salmonella typhimurium knockout strains STM3120-, STM3120-ttrA- and STM3120-ttrA-ADI-, 10\hat{ }6 CFU per mouse.
(A) relative body weight. N=4 for all groups. Whiskers represent SE.
(B) Bacteria tumor homing capacity. Mice were sacrificed 8 days post injections. Tumors were harvested, suspended in LB and seeded on LB plate. CFU count was normalized to tissue weight and suspension volume.

FIGS. 13A-B. Illustration showing the design of the neoantigen cassette to be inserted into the bacterial genome. The cassette is composed of homology arms to the endogenous ompA gene, a neoantigen sequence to be inserted in the coding sequence (CDS) of the gene to obtain presentation on the bacterial cell wall. Downstream to the coding sequence two more versions of the neoantigen are added, the first includes the MISSSSIS secretion signal (type 3 secretion system—T3SS) and the last one includes the pelB secretion system (type 2 secretion system—T2SS). For each type of neoantigen that will be checked (ADPGK for MC38 tumors, E7 for TC-1 tumors and SIINFEKL (SEQ ID NO: 11) for B16-Ova tumors), such a cassette will be inserted in the genome.

FIG. 14 is a graph illustrating functional testing of human IL18 secreted by STM3120. Supernatant of exponentially growing bacteria was concentrated by 5 kDa centrifugal filter tube. HEK-blue IL18 reporter cells were incubated for 24 hours with different dilutions of recombinant human IL18 or bacteria sup. Cells' pre conditional medium was subjected to Quanti-blue colorimetric enzyme assay, and signal was read 3 hrs later.

FIGS. 15A-B. In vitro and in vivo validation of luciferase induced by Aspirin in STM3120.
(A) Exponentially growing STM3120 expressing luciferase under induction of Aspirin were incubated with different concentration of Aspirin. Luminescence was quantified in duplicates over 24 hours. RLU—relative luminescence units.
(B) Mice bearing MC38 sub-cutaneous tumors (>100 mm\hat{ }3) were injected I.V with STM3120 expressing luciferase, 10\hat{ }6 CFU per mouse. Twenty four hours post injection, mice were gavaged with either vehicle control or Aspirin 25 mg/kg and luminescence was read 5 hours later. Color bar represents relative luminescence unit. range: 0-1×10\hat{ }6 RLU.

FIG. 16 is a graph illustrating the effect of aspirin on the growth of Salmonella typhimurium (STM3120).

STM3120 (STM) bacteria and STM3120 bacteria harboring a plasmid with Aspirin inducible luciferase expression (STMpSalux) were grown with 200 uM Aspirin (+Asp) or without Aspirin (−Asp) and OD was measured to reflect their growth rate. LB—Luria Broth medium with no bacteria.

FIGS. 17A-B. The effect of Salmonella ghosts on systemic toxicity and tumor growth.

Mice bearing MC38 sub-cutaneous tumors (˜100 mm\hat{ }3) were injected I.V with STM3120 expressing the MC38 neoantigen ADPGK (STM-ADPGK), either live or paraformaldehyde-killed (ghost).
(A) relative body weight. N=6-7 for all groups. Whiskers represent SE.
(B) Tumor growth in mice treated with I.V injection of 3*10\hat{ }7 ghosts (N=7) was compared to No treatment group (N=3), or mice treated with 150 ug aPD1 once a week (N=4). In aPD1 cohort 1 mouse was fully cured. In the ghost cohort, 3 mice were fully cured.

FIGS. 18A-B. Attenuation of STM3120 by deleting antibiotic resistance or infectivity associated genes.

C57BL/6 mice were injected with 10\hat{ }6 MC38 cells in the right flank. When tumors reached a volume of ˜100 mm\hat{ }3, mice receive intravenous (i.v.) injections with 10\hat{ }6 CFU of STM3120- or STM3120-aac6-deleted) or STM3120-ttrA- or STM3120-ttrA-adI-. Mice received weekly administration of 75/150 ug anti-PD1, i.p.
A. Graph shows the relative body weight of the mice following injection of bacteria (day 0). It was noted that the strains with additional attenuations recovered faster that STM3120-.
B. Graph shows the homing capacity of the bacteria. After 9 days, tumors were resected and vigorously shaken in 1 ml LB and a metal ball. Supernatant was seeded on LB plates and colonies were counted following 24 hrs incubation at 37° C. CFU was normalized to the dilution factor and tissue mass. A mild reduction in homing capacity was detected using the attenuated strains.

### DESCRIPTION OF SPECIFIC EMBODIMENTS OF THE INVENTION

The present invention, in some embodiments thereof, relates to bacterial vaccines which may be manipulated to express disease-associated antigens.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In vivo therapeutic cancer vaccine strategies based on bacterial vectors that directly deliver antigens or nucleic acids encoding antigens to the cytosol of APCs, have been developed in academic laboratories and pharmaceutical industry due to their ease of use. Typically, the bacteria are genetically modified to express (and even secrete) the disease antigen. Alternatively, the bacteria may be used to deliver plasmid cDNA which encode the disease antigen to the immune system.

The present inventors have now conceived of a novel vaccine which includes bacteria engineered for reduced virulence, toxicity, pathogenicity, tumor-homing, and/or antibiotic resistance.

According to one embodiment, the bacteria are genetically engineered (i.e. modified) to increase tumor-homing.

As used herein, “engineered for tumor-homing” is meant to include genetically engineered bacterium which results in at least one of: increased numbers of colony forming units within the solid tumor compared to its parental strain; increased serum half-life compared to its parental strain; increased numbers of colony forming units within the solid tumor compared to its parental strain; and reduced immune elimination following repeated dosing compared to its parental strain.

The bacteria are genetically modified to express disease associated antigens. These vaccines are referred to herein as Personalized Anti-Cancer Microbiome-Assisted Vaccination (PACMAN).

Surprisingly, as is illustrated herein and in the figures and examples section, the present inventors show that it is possible to genetically modify tumor homing bacteria to express tumor antigens. The genetically modified bacteria serve two purposes 1) as a targeting vehicle-homing to the tumor site and 2) as an adjuvant, stimulating the immune system.

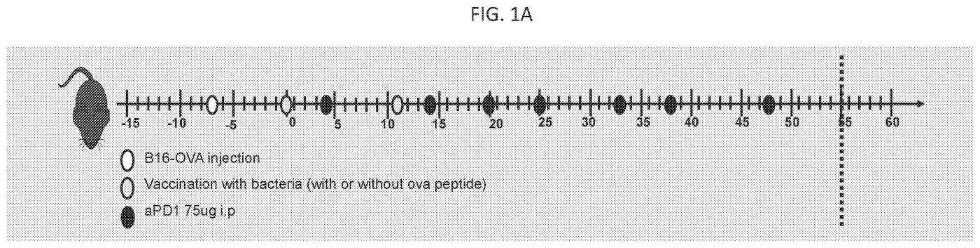

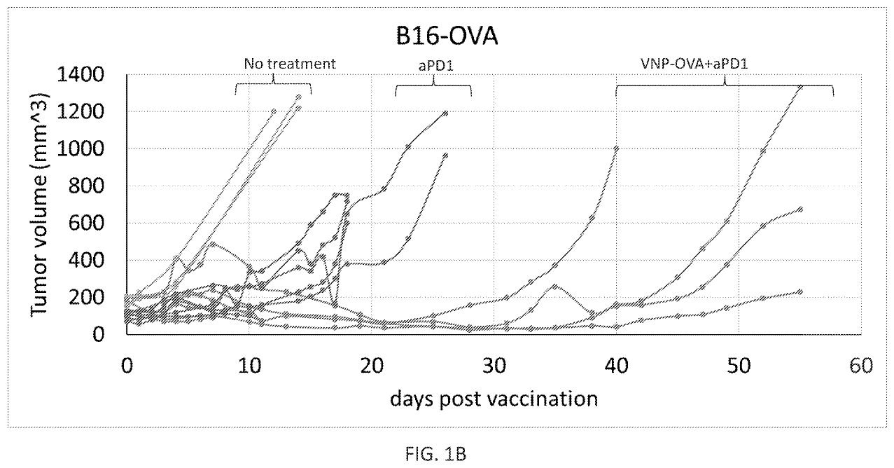

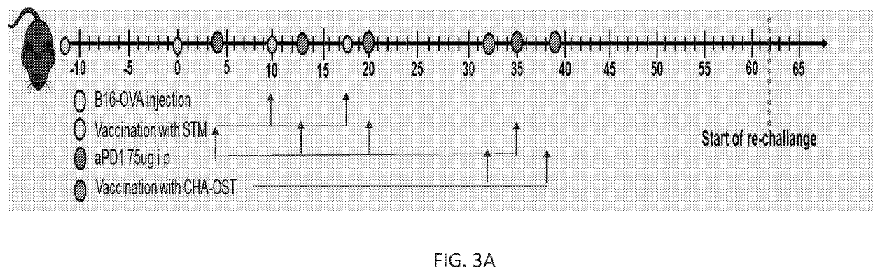

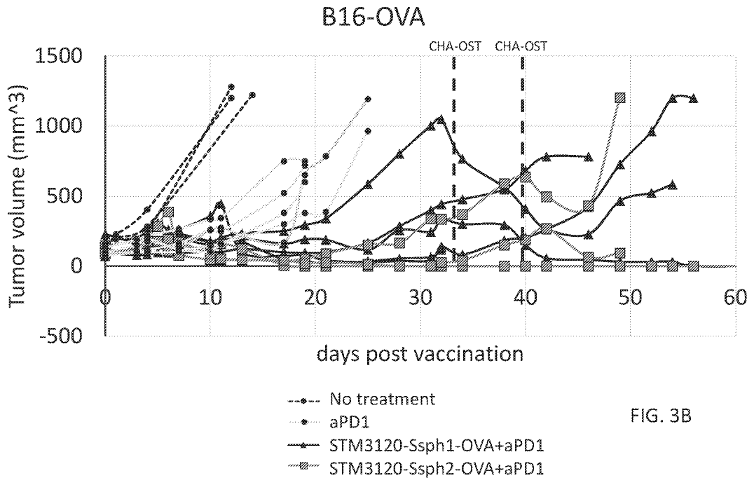

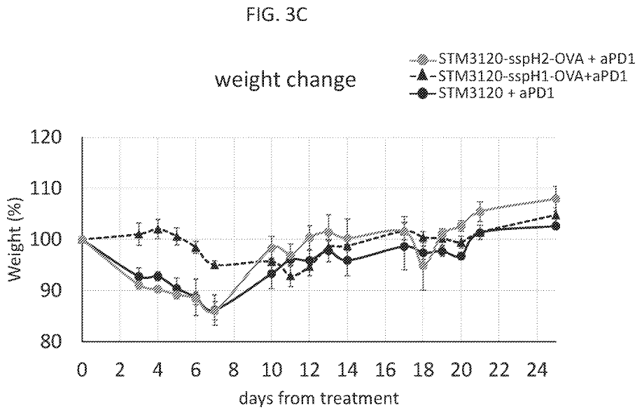

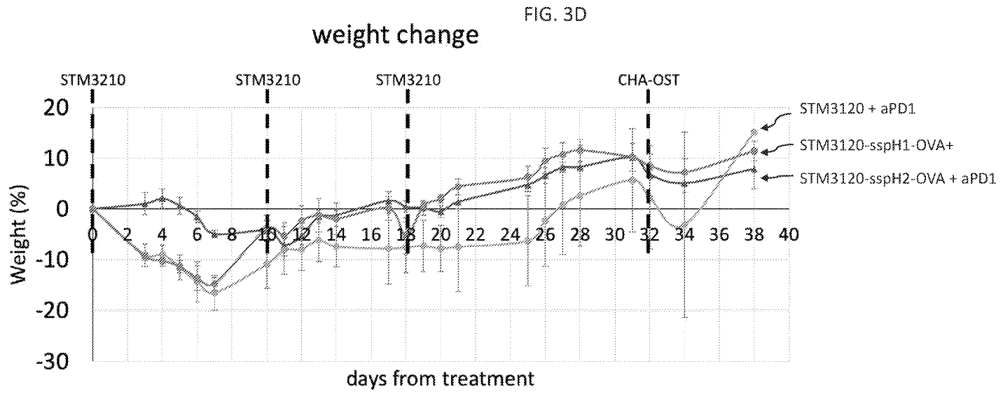

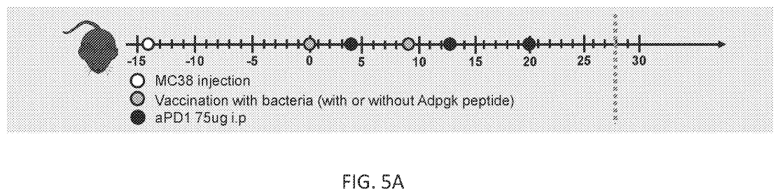

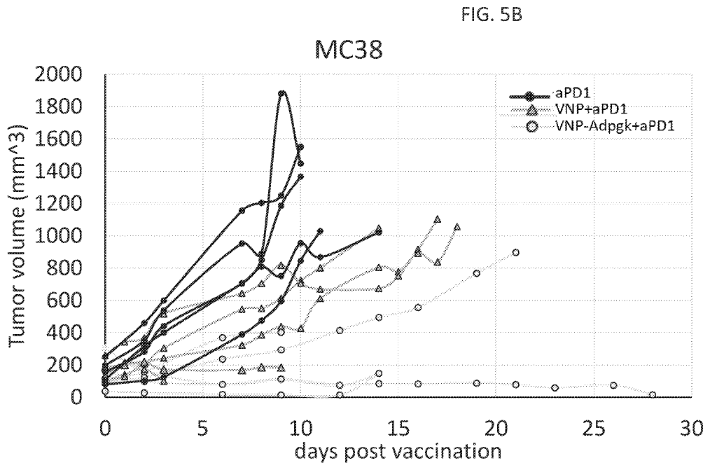

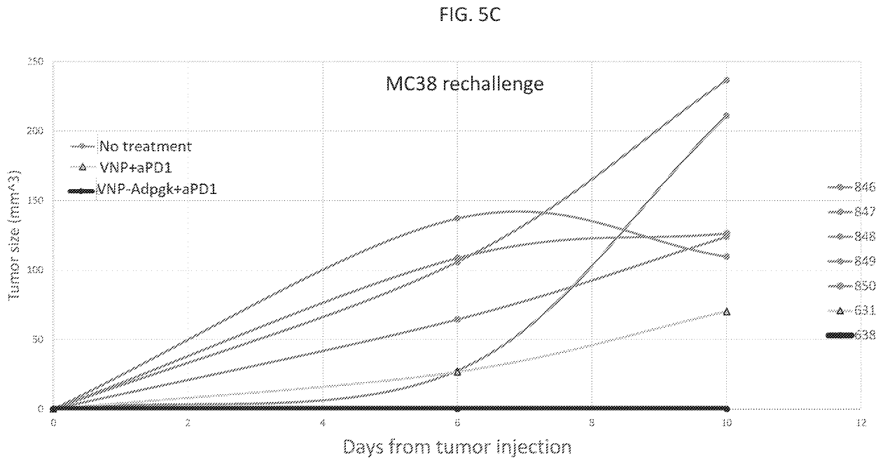

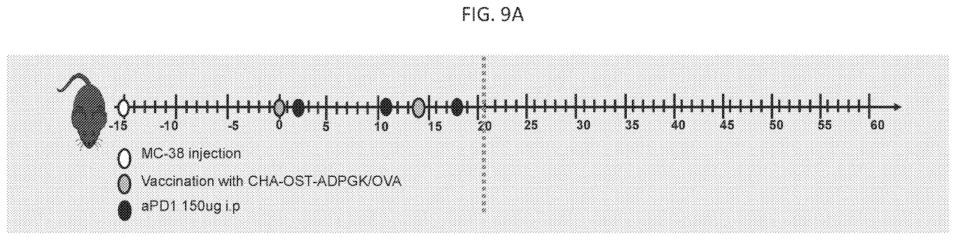

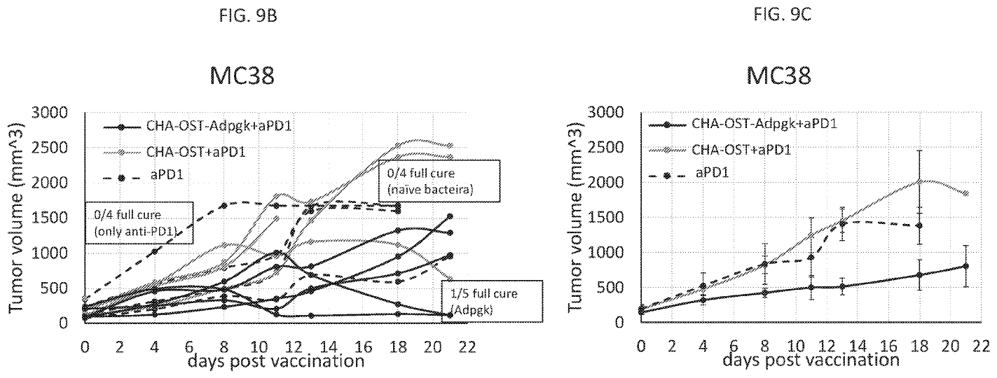

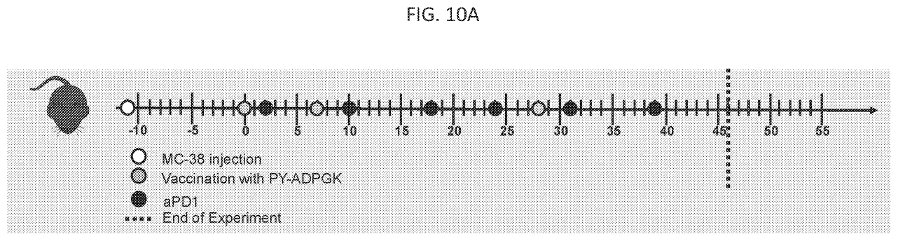

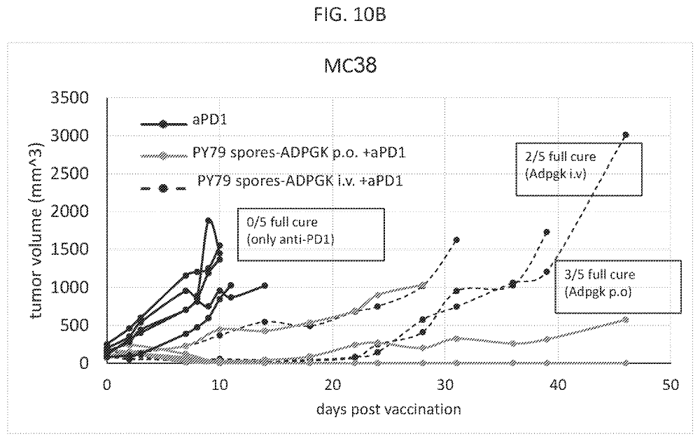

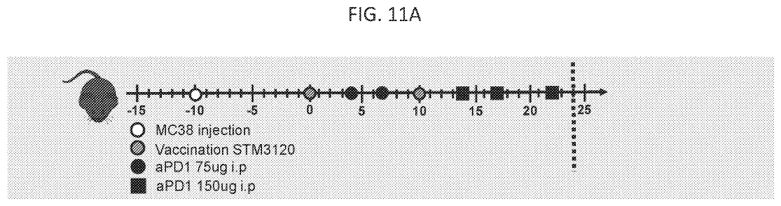

The inventors demonstrated the ability to produce effective vaccines using a number of different bacteria including Salmonella typhimurium (FIGS. 1A-B, 2A-G, 3A-D, 4A-B, 5A-C), P. aeruginosa (FIGS. 9A-B) and B. subtilis (FIGS. 10A-B). The bacteria were genetically modified to express various tumor specific antigens including OVA (FIGS. 1A-B, 2A-G, 3A-D, 4A-B) and ADPGK (FIGS. 5A-C, 9A-B, 10A-B and 11A-B), as well as showing that alternate administration of different bacterial vaccines can overcome acquired immunity (see FIGS. 3A-C).

Thus, according to an aspect of the present invention there is provided a vaccine comprising tumor-homing bacteria which are genetically modified to express at least one cancer-associated antigen.

As used herein, the term “vaccine” refers to a pharmaceutical preparation (pharmaceutical composition) that upon administration induces an immune response, in particular a cellular immune response, which recognizes and attacks a target cell (e.g., a cancer cell). Preferably, the vaccine results in the formation of long-term immune memory towards the targeted antigen. The vaccine of the present invention preferably also includes a pharmaceutically acceptable carrier (e.g., a liquid composition which carries the bacteria). In one embodiment, the carrier is one which retains the viability of the bacteria.

The bacteria of this aspect of the present invention may be Gram positive or Gram negative bacteria or may be a combination of both.

The term “Gram-negative bacteria” are bacteria that do not retain the crystal violet dye in the Gram staining protocol. The bacteria (e.g., Gram negative bacteria) may be aerobic or anaerobic bacteria.

In one embodiment, the bacteria are capable of homing to a tumor site.

According to another embodiment, the bacteria are capable of colonizing a tumor. According to still another embodiment, the bacteria are capable of homing to (e.g., reaching the tumor site following i.v. administration and colonizing the tumor).

In another embodiment, the bacteria (e.g., Gram negative bacteria) which are capable of homing to (and/or colonizing) a tumor are present in a tumor microbiome of the subject. Examples of relevant bacteria present in a tumor microbiome are provided but not limited to the following: WO2021/205444, WO2022/175951 and WO2022/175952.

The term “tumor microbiome” refers to the totality of microbes (bacteria, fungae, protists), their genetic elements (genomes) in a defined environment, e.g., within the tumor of a host. In a particular embodiment, the microbiome refers only to the totality of bacteria in a defined environment, e.g., within the tumor of a host. The tumor may be a primary tumor or a secondary tumor (i.e. metastasized tumor).

Examples of bacteria known to be present in a breast tumor microbiome are provided in WO2021/205444, WO2022/175951 and WO2022/175952, the contents of which are incorporated herein by reference. Such bacteria may be particularly relevant for use in vaccines for treating breast cancer.

In some embodiments, the bacteria is a gram negative bacteria belonging to a genus from the list consisting of: Acidovorax, Acinetobacter, Agrobacterium, Alcaligenes, Bacteroides, Citrobacter, Devosia, Eikenella, Enhydrobacter, Enterobacter, Erwinia, Escherichia/Shigella, Fusobacterium, Kaistobacter, Klebsiella, Leptotrichia, Luteimonas, Massilia, Methylobacterium, Neisseria, Paracoccus, Prevotella, Proteus, Pseudomonas, Psychrobacter, Ralstonia, Roscomonas, Selenomona, Shewanella, Sphingobium, Sphingomonas, Tepidimonas, Treponema, Veillonella, Wautersiella, Xanthomonas.

In some embodiments, the bacteria is a gram-negative bacterium belonging to a genus from the list consisting of: Acidovorax, Acinetobacter, Bacteroides, Citrobacter, Fusobacterium, Klebsiella, Neisseria, Prevotella, Pseudomonas, Treponema, Veillonella. In some embodiments, the bacteria is a gram-negative bacterium belonging to a genus from the list consisting of: Citrobacter, Klebsiella, Neisseria, Pseudomonas.

In some embodiments, the bacteria is a gram-negative bacteria belonging to a species from the list consisting of: Acidovorax temperans, Acinetobacter radioresistens, Acinetobacter ursingii, Alcaligenes faecalis, Bacteroides dorei, Citrobacter Freundii, Eikenella corrodens, Enhydrobacter acrosaccus, Enterobacter acrogenes, Enterobacter asburiac, Enterobacter cloacae, Fusobacterium nucleatum, Kaistobacter Unknown, Klebsiella oxytoca, Klebsiella pneumoniae, Massilia timonae, Methylobacterium mesophilicum, Methylobacterium organophilum, Neisseria macacae, Neisseria subflava, Paracoccus aminovorans, Paracoccus chinensis, Paracoccus marcusii, Prevotella melaninogenica, Prevotella tannerac, Prevotella Unknown, Proteus mirabilis, Pseudomonas argentinensis, Pseudomonas bactica, Pseudomonas mendocina, Pseudomonas viridiflava, Ralstonia mannitolilytica. Roscomonas mucosa, Shewanella decolorationis, Sphingomonas desiccabilis, Sphingomonas yanoikuyac, Sphingomonas yunnanensis, Treponema socranskii, Veillonella dispar, Veillonella parvula and Xanthomonas arboricola. In a particularly preferred embodiment, the bacteria is a gram-negative bacterium belonging to a species from the list consisting of: Acidovorax temperans, Acinetobacter radioresistens, Bacteroides dorei, Fusobacterium nucleatum, Klebsiella pneumoniae and Veillonella parvula. In another embodiment, bacteria is a gram-negative bacterium belonging to a species selected from the list consisting of: Bacteroides dorei, Fusobacterium nucleatum and Klebsiella pneumoniae.

According to a particular embodiment, the bacteria is considered pathogenic before attenuation. Examples include, but are not limited to, Salmonella spp., Yersinia spp., Bordetella spp., Escherichia coli, Shigella spp., Burkholderia mallei, Burkholderia pseudomallei and Pseudomonas aeruginosa. In certain embodiments, the attenuated bacteria are Salmonella enterica.

In some embodiments, the bacteria is Salmonella typhimurium—e.g., the Salmonella typhimurium attenuated strain VNP20009, Salmonella typhimurium 14028 strain STM3120 (also referred to herein as STM3120 strain; see for example Arrach et al., Cancer Res. 2010 Mar. 15; 70 (6): 2165-2170. doi: 10.1158/0008-5472.CAN-09-4005), Salmonella typhimurium 14028 strain STM1414, Pseudomonas aeruginosa (strain CHA-OST) and/or Bacillus subtillis (strain PY79). In some embodiments, the Salmonella typhimurium is Salmonella typhimurium VPN20009. In some embodiments, the Salmonella typhimurium VPN20009 is a Salmonella typhimurium VPN20009 3120. In some embodiments, the Salmonella typhimurium has no mutation or reduction in expression conferring purine-auxotrophy (e.g., mutation affecting the pur I gene)

The term “isolated” or “enriched” encompasses bacteria that has been (1) separated from at least some of the components with which it was associated when initially produced (whether in nature or in an experimental setting), and/or (2) produced, prepared, purified, and/or manufactured by the hand of man. Isolated microbes may be separated from at least about 10%, about 20%, about 30%, about 40%, about 50%, about 60%, about 70%, about 80%, about 90%, or more of the other components with which they were initially associated. In some embodiments, isolated microbes are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a microbe or other material that has been separated from at least some of the components with which it was associated either when initially produced or generated (e.g., whether in nature or in an experimental setting), or during any time after its initial production. A microbe or a microbial population may be considered purified if it is isolated at, or after production, such as from a material or environment containing the microbe or microbial population, and a purified microbe or microbial population may contain other materials up to about 10%, about 20%, about 30%, about 40%, about 50%, about 60%, about 70%, about 80%, about 90%, or above about 90% and still be considered “isolated.” In some embodiments, purified microbes or microbial population are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components thereof are generally purified from residual habitat products.

In certain embodiments, at least 50%, 55%, 60%, 65%, 70%, 75%, 80%, 85%, 90%, 95%, 96%, 97%, 98%, 99% of the bacteria in the vaccine are of a genus, species or strain disclosed herein or in WO2021/205444, WO2022/175951 and WO2022/175952, or selected from those specifically mentioned above.

According to a specific embodiment, the genome of the bacteria comprises a 16S rRNA sequence at least 90%, 91%, 92%, 93%, 94%, 95%, 96%, 97%, 98% 99%, 99.1%, 99.2%, 99.3%, 99.4%, 99.5%, 99.6%, 99.7%, 99.8%, 99.9%, 99.95% identical to any one of the sequences disclosed herein or in WO2021/205444, WO2022/175951 and WO2022/175952.

As used herein, “percent homology”, “percent identity”, “sequence identity” or “identity” or grammatical equivalents as used herein in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89 (22): 10915-9].

Percent identity can be determined using any homology comparison software, including for example, the BlastN software of the National Center of Biotechnology Information (NCBI) such as by using default parameters.

Other exemplary sequence alignment programs that may be used to determine % homology or identity between two sequences include, but are not limited to, the FASTA package (including rigorous (SSEARCH, LALIGN, GGSEARCH and GLSEARCH) and heuristic (FASTA, FASTX/Y, TFASTX/Y and FASTS/M/F) algorithms, the EMBOSS package (Needle, stretcher, water and matcher), the BLAST programs (including, but not limited to BLASTN, BLASTX, TBLASTX, BLASTP, TBLASTN), megablast and BLAT. In some embodiments, the sequence alignment program is BLASTN. For example, 95% homology refers to 95% sequence identity determined by BLASTN, by combining all non-overlapping alignment segments (BLAST HSPs), summing their numbers of identical matches and dividing this sum with the length of the shorter sequence.

In some embodiments, the sequence alignment program is a basic local alignment program, e.g., BLAST. In some embodiments, the sequence alignment program is a pairwise global alignment program. In some embodiments, the pairwise global alignment program is used for protein-protein alignments. In some embodiments, the pairwise global alignment program is Needle. In some embodiments, the sequence alignment program is a multiple alignment program. In some embodiments, the multiple alignment program is MAFFT. In some embodiments, the sequence alignment program is a whole genome alignment program. In some embodiments, the whole genome alignment is performed using BLASTN. In some embodiments, BLASTN is utilized without any changes to the default parameters.

According to some embodiments of the invention, the identity is a global identity, i.e., an identity over the entire nucleic acid sequences of the invention and not over portions thereof.

Methods of qualifying which bacteria are present in a tumor microbiome are described herein below. Care should be taken to take a sufficient number of measurements when analyzing which microbes are present in the microbiome to minimize and control for contaminations.

In some embodiments, determining a presence of one or more bacteria or components or products thereof comprises determining a level or set of levels of one or more DNA sequences. In some embodiments, one or more DNA sequences comprises any DNA sequence that can be used to differentiate between different bacterial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

In some embodiments, a microbiota sample (e.g., tumor sample) is directly assayed for a presence, a level or set of levels of one or more DNA sequences. In some embodiments, DNA is isolated from a tumor microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QIAamp DNA Stool Mini Kit (Qiagen, Valencia, Calif.).

In some embodiments, a presence, a level or set of levels of one or more DNA sequences is determined by amplifying DNA sequences using PCR (e.g., standard PCR, semi-quantitative, or quantitative PCR). In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a presence, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a presence, a level or set of levels of one or more types of microbes or components or products thereof comprises determining a presence, a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis.

In some embodiments, determining a presence, a level or set of levels of one or more types of microbes or components or products thereof comprises determining a presence, a level or set of levels of one or more microbial polypeptides. Methods of quantifying polypeptide levels are well known in the art and include but are not limited to Western analysis and mass spectrometry. These and all other basic polypeptide detection procedures are described in Ausebel et al.

In some embodiments, determining a presence, a level or set of levels of one or more types of microbes or components or products thereof comprises determining a presence, a level or set of levels of one or more microbial metabolites. In some embodiments, levels of metabolites are determined by mass spectrometry. In some embodiments, levels of metabolites are determined by nuclear magnetic resonance spectroscopy. In some embodiments, levels of metabolites are determined by enzyme-linked immunosorbent assay (ELISA). In some embodiments, levels of metabolites are determined by colorimetry. In some embodiments, levels of metabolites are determined by spectrophotometry.

In certain embodiments, the vaccine comprises at least 1×10³ colony forming units (CFUs), 1×10⁴ colony forming units (CFUs), 1×10⁵ colony forming units (CFUs), 1×10⁶ colony forming units (CFUs), 1×10⁷ colony forming units (CFUs), 1×10⁸ colony forming units (CFUs), 1×10⁹ colony forming units (CFUs), 1×10¹⁰ colony forming units (CFUs) of bacteria of a family/genus/species/strain listed herein. In certain embodiments, the vaccine comprises less than 1×10⁹ colony forming units (CFUs).

Methods for producing bacteria may include three main processing steps. The steps are: organism banking, organism production, and preservation.

For banking, the strains included in the bacteria may be (1) isolated directly from a specimen or taken from a banked stock, (2) optionally cultured on a nutrient agar or broth that supports growth to generate viable biomass, and (3) the biomass optionally preserved in multiple aliquots in long-term storage.

In embodiments using a culturing step, the agar or broth may contain nutrients that provide essential elements and specific factors that enable growth. An example would be a medium composed of 20 g/L glucose, 10 g/L yeast extract, 10 g/L soy peptone, 2 g/L citric acid, 1.5 g/L sodium phosphate monobasic, 100 mg/L ferric ammonium citrate, 80 mg/L magnesium sulfate, 10 mg/L hemin chloride, 2 mg/L calcium chloride, 1 mg/L menadione. Other examples would be a medium composed of 10 g/L beef extract, 10 g/L peptone, 5 g/L sodium chloride, 5 g/L dextrose, 3 g/L yeast extract, 3 g/L sodium acetate, 1 g/L soluble starch, and 0.5 g/L L-cysteine HCl, at pH 6.8. A variety of microbiological media and variations are well known in the art (e.g., R. M. Atlas, Handbook of Microbiological Media (2010) CRC Press). Culture media can be added to the culture at the start, may be added during the culture, or may be intermittently/continuously flowed through the culture. The strains in the vaccine may be cultivated alone, as a subset of the microbial composition, or as an entire collection comprising the microbial composition. As an example, a first strain may be cultivated together with a second strain in a mixed continuous culture, at a dilution rate lower than the maximum growth rate of either cell to prevent the culture from washing out of the cultivation.

The inoculated culture is incubated under favorable conditions for a time sufficient to build biomass. For microbial compositions for human use this is often at 37° C. temperature, pH, and other parameter with values similar to the normal human niche. The environment may be actively controlled, passively controlled (e.g., via buffers), or allowed to drift. For example, for anaerobic bacterial compositions, an anoxic/reducing environment may be employed. This can be accomplished by addition of reducing agents such as cysteine to the broth, and/or stripping it of oxygen. As an example, a culture of a bacterial composition may be grown at 37° C., pH 7, in the medium above, pre-reduced with 1 g/L cysteine-HCl.

When the culture has generated sufficient biomass, it may be preserved for banking. The organisms may be placed into a chemical milieu that protects from freezing (adding ‘cryoprotectants’), drying (‘lyoprotectants’), and/or osmotic shock (osmoprotectants′), dispensing into multiple (optionally identical) containers to create a uniform bank, and then treating the culture for preservation. Containers are generally impermeable and have closures that assure isolation from the environment. Cryopreservation treatment is accomplished by freezing a liquid at ultra-low temperatures (e.g., at or below −80° C.). Dried preservation removes water from the culture by evaporation (in the case of spray drying or ‘cool drying’) or by sublimation (e.g., for freeze drying, spray freeze drying). Removal of water improves long-term microbial composition storage stability at temperatures elevated above cryogenic. If the microbial composition comprises, for example, spore forming species and results in the production of spores, the final composition may be purified by additional means such as density gradient centrifugation preserved using the techniques described above. Microbial composition banking may be done by culturing and preserving the strains individually, or by mixing the strains together to create a combined bank. As an example of cryopreservation, a microbial composition culture may be harvested by centrifugation to pellet the cells from the culture medium, the supernatant decanted and replaced with fresh culture broth containing 15% glycerol. The culture can then be aliquoted into 1 mL cryotubes, sealed, and placed at −80° C. for long-term viability retention. This procedure achieves acceptable viability upon recovery from frozen storage.

Microbial production may be conducted using similar culture steps to banking, including medium composition and culture conditions. It may be conducted at larger scales of operation, especially for clinical development or commercial production. At larger scales, there may be several subcultivations of the microbial composition prior to the final cultivation. At the end of cultivation, the culture is harvested to enable further formulation into a dosage form for administration. This can involve concentration, removal of undesirable medium components, and/or introduction into a chemical milieu that preserves the microbial composition and renders it acceptable for administration via the chosen route. After drying, the powder may be blended to an appropriate potency, and mixed with other cultures and/or a filler such as microcrystalline cellulose for consistency and ease of handling, and the bacterial composition formulated as provided herein.

In certain aspects, provided are vaccines (i.e., bacterial compositions) for administration to subjects. In some embodiments, the bacteria are combined with additional active and/or inactive materials in order to produce a final product, which may be in single dosage unit or in a multi-dose format.

The bacteria present in the vaccine may be viable (e.g., capable of propagating when cultured in the appropriate medium, or inside the body, following administration).

In another embodiment, the bacteria present in the vaccine are non-viable. An example of a non-viable bacteria is a bacterial ghost. In another embodiment, the vaccine may be a formulation of viable and non-viable bacteria.

Bacterial ghosts are a specific type of inactivated bacteria, namely cell envelopes created by controlled bacterial cell lysis. One method is by using a cell lysing agent such as paraformaldehyde. Another method is the expression of a recombinant lysis gene obtained from a phage, such as gene E from phage.phi.X174. E is a small protein that forms a pore in the bacterial cell membrane, allowing all cytoplasmic content to flow out of the bacteria, thereby killing the bacteria but leaving the cell with a preserved cellular morphology including all cell surface structures. Other methods for creating bacterial ghosts could be performed by one of ordinary skill in the art (see for example Langemann et al., Bioengineered Bugs 1:5, 326-336; September/October 2010), the contents of which are incorporated herein by reference. Bacterial ghosts exhibit intrinsic adjuvant properties and trigger an enhanced humoral and cellular immune response to the target antigen. Preparations containing bacterial proteins associated with the outer membrane can be made using well-known in the art techniques. One such technique is described in U.S. Pat. No. 6,432,412. These preparations are referred herein as “membrane fractions”.

In still another embodiment, the bacteria are attenuated such that they are not capable of causing disease.

Thus, according to another aspect there is provided a bacteria of the species Salmonella enterica genetically modified to express a reduced amount or a less active product of at least one gene selected from the group consisting of arginine deaminase (adI), L-asparaaginase II (asnB), Aminoglycoside (3″) (9) adenylyltransferase (aadA), AAC (6′)-Iaa (aac6) and Tetrathionate reductase A (ttrA) as compared to non-attenuated bacteria of the species Salmonella enterica. Thus, the attenuated bacteria may have a null mutation in at least one, two, three or all of the above-mentioned genes. In some embodiments, the Salmonella typhimurium bacteria have two or more genomic mutations, deletions or expression reduction of a gene selected from the list consisting of: AAC (6′)-Iaa (aac6) arginine deaminase (adI), L-asparaaginase II (asnB). Aminoglycoside (3″) (9) adenylyltransferase (aadA), and Tetrathionate reductase A (ttrA) compared to non-attenuated bacteria of the species Salmonella enterica. In some embodiments, the Salmonella typhimurium bacteria have three or more gene genomic mutations, deletions or expression reduction of a gene selected from the list consisting of: AAC (6′)-Iaa (aac6) arginine deaminase (adI). L-asparaaginase II (asnB). Aminoglycoside (3″) (9) adenylyltransferase (aadA), and Tetrathionate reductase A (ttrA) compared to non-attenuated bacteria of the species Salmonella enterica. In some embodiments, the Salmonella typhimurium have four or more gene genomic mutations, deletions or expression reduction of a gene selected from the list consisting of: AAC (6′)-Iaa (aac6) arginine deaminase (adI). L-asparaaginase II (asnB), Aminoglycoside (3″) (9) adenylyltransferase (aadA), and Tetrathionate reductase A (ttrA) compared to non-attenuated bacteria of the species Salmonella enterica. In some embodiments, the Salmonella typhimurium includes genomic mutation, deletions, or expression reduction in one of the following genes: arginine deiminase (adI) and tetrathionate reductase A (ttrA). In another embodiments, although Salmonella enterica is referred to in this group of embodiments, it is to be recognized that any bacteria having homologous genes can be similarly modified with genomic mutation, deletions, or expression reduction as described herein.

In some embodiments, the genomic mutations mentioned above are used for chromosomal integration of polynucleotide cassettes.

The attenuated bacteria may be of the serotype Typhimurium (e.g., strain STM3120 (i.e. having a deletion in STM3120).

The term “attenuated” refers to a bacteria rendered to be less virulent compared to the native bacteria, thus, becoming harmless or less virulent. Preferably, the ability to home to a tumor is not reduced by the attenuation, such that homing ability is not reduced by more than 80%, more preferably 70%, more preferably 60% more preferably 50%, more preferably 40%, more preferably 30%, more preferably 20%, more preferably 10% as compared to non-attenuated (native bacteria) following i.v. administration (e.g., in a mouse model).

The attenuated bacteria may be attenuated by making the bacteria deficient in one or more target genes that are associated with pathogenicity. Suitable genes may include but are not limited to at least one of the following:
arginine deiminase (adI); Alternative names: NP_463327.1 or locus tag STM4467;
L-asparaginase II (ansB); Alternative names: NP_462022.1 or locus tag STM3106;
Aminoglycoside resistance protein (aadA): Alternative names: NP_460230.1 or locus tag STM1264;
AAC (6′)-Iaa (aac6); Alternative names: NP_460578.1 or locus tag STM1619;
Tetrathionate reductase A (ttrA). Alternative names: NP_460348.1 or locus tag: STM1383.

As well as the above mentioned genes, the attenuated bacteria may also have a mutation in STM3120 (STM3120 putative LysR family transcriptional regulator [Salmonella enterica subsp. enterica serovar Typhimurium str. LT2) (STM3120; Gene ID 1254643).

The bacteria may be made deficient of one or more of the above-mentioned target genes by a method that includes deleting at least a portion of the target gene by recombination and insertion of a selectable marker in place of the deleted portion of the target gene. Subsequently, the selectable marker may be deleted in order to prepare a markerless bacterium that is deficient in the target gene.

Suitable methods for preparing the markerless bacteria that are deficient in the one or more target genes may include recombineering systems. The recombineering systems may include: (a) a mobilizable recombineering vector that expresses protein components for facilitating homologous recombination; and (b) a linear DNA molecule that is configured for recombining at a target gene and replacing at least a portion of the target gene with a selectable marker that is flanked by recombinase recognition target sequences. After the linear DNA molecule is recombined at the target sequence, a recombinase that recognizes the recombinase recognition target sequences may be expressed in order to recombine the target sequences and remove the selectable marker that is flanked by recombinase recognition target sequences.

The phrase “heterologous protein” or “recombinantly produced heterologous protein” refers to a peptide or protein of interest produced using cells that do not have an endogenous copy of DNA able to express the peptide or protein of interest. The cells produce the peptide or protein because they have been genetically altered by the introduction of the appropriate nucleic acid sequences. The recombinant peptide or protein will not be found in association with peptides or proteins and other subcellular components normally associated with the cells producing the peptide or protein.

As mentioned, according to some embodiments, the bacteria express a heterologous or non-native protein or peptide (e.g., antigen) which is capable of inducing an antigen-specific immune response in a subject. In some embodiments, the bacteria of the vaccine disclosed herein express at least one cancer-associated antigen.

The term cancer-associated antigen (also referred to as a tumor antigen) refers to an antigenic substance (e.g. peptide) produced in tumor cells which triggers an immune response in the host. In one embodiment the immune response is an increase in the production of CD8+ T cells and/or CD4+T cells.

Cancer-associated antigens are typically short peptides corresponding to one or more antigenic determinants of a protein which is expressed (e.g., selectively) in a tumor cell. The cancer-associated antigen typically binds to a class I or II MHC receptor thus forming a ternary complex that can be recognized by a T-cell bearing a matching T-cell receptor binding to the MHC/peptide complex with appropriate affinity. Peptides binding to MHC class I molecules are typically about 8-14 amino acids in length. T-cell epitopes that bind to MHC class II molecules are typically about 12-30 amino acids in length. In the case of peptides that bind to MHC class II molecules, the same peptide and corresponding T cell epitope may share a common core segment but differ in the overall length due to flanking sequences of differing lengths upstream of the amino-terminus of the core sequence and downstream of its carboxy terminus, respectively. A T-cell epitope may be classified as an antigen if it elicits an immune response.

The antigens for cancers can be antigens from testicular cancer, ovarian cancer, brain cancer such as glioblastoma, pancreatic cancer, melanoma, lung cancer, prostate cancer, hepatic cancer, breast cancer, rectal cancer, colon cancer, esophageal cancer, gastric cancer, renal cancer, sarcoma, neuroblastoma, Hodgkin's and non-Hodgkin's lymphoma and leukemia.

In one embodiment, the cancer-associated antigen is a cancer testis antigen (e.g., a member of the melanoma antigen protein (MAGE) family, Squamous Cell Carcinoma-1 (NY-ESO-1), BAGE (B melanoma antigen), LAGE-1 antigen, Brother of the Regulator of Imprinted Sites (BORIS) and members of the GAGE family).

In another embodiment, the cancer-associated antigen is derived from MART-1/Melan-A protein e.g. (MARTI MHC class I peptides (Melan-A: 26-35 (L27), ELAGIGILTV; SEQ ID NO: 1) and MHC class II peptides (Melan-A: 51-73 (RR-23) RNGYRALMDKSLHVGTQCALTRR; SEQ ID NO: 2).

In another embodiment, the cancer-associated antigen is derived from glycoprotein 70, glycoprotein 100 (gp100: 25-33 (MHC class I (EGSRNQDWL—SEQ ID NO: 7)) or gp100: 44-59 MHC class II (WNRQLYPEWTEAQRLD—SEQ ID NO: 8) peptides).

In still another embodiment, the cancer-associated antigen is derived from tyrosinase, tyrosinase-related protein 1 (TRP1), tyrosinase-related protein 2 (TRP-2) or TRP-2/INT2 (TRP-2/intron2).

In still another embodiment, the cancer-associated antigen comprises MUT30 (mutation in Kinesin family member 18B, Kif18b-PSKPSFQEFVDWENVSPELNSTDQPFL—SEQ ID NO: 9) or MUT44 (cleavage and polyadenylation specific factor 3-like, Cpsf31 EFKHIKAFDRTFANNPGPMVVFATPGM—SEQ ID NO: 10).

In still another embodiment, the cancer-associated antigen is derived from stimulator of prostatic adenocarcinoma-specific T cells-SPAS-1.

In still another embodiment, the cancer-associated antigen is derived from human telomerase reverse transcriptase (hTERT) or hTRT (human telomerase reverse transcriptase).

In still another embodiment, the cancer-associated antigen is derived from ovalbumin (OVA) for example OVA₂₅₇₋₂₆₄ MHCI H-2Kb (SIINFEKL—SEQ ID NO: 11) and OVA₃₂₃₋₃₃₉ MHCII I-A (d) (ISQAVHAAHAEINEAGR SEQ ID NO: 12), a RAS mutation, mutant oncogenic forms of p53 (TP53) (p53mut (peptide antigen of mouse mutated p53_{R172H} sequence VVRHCPHHER—SEQ ID NO: 4 (human mutated p53_{R175H} sequence EVVRHCPHHE—SEQ ID NO: 5)), or from BRAF-V600E peptide (GDFGLATEKSRWSGS—SEQ ID NO: 13).

According to a particular embodiment, the cancer associated antigen is set forth in SEQ ID NO: 11.

In still another embodiment, the cancer-associated antigen is a breast cancer associated disease antigen including but not limited to α-Lactalbumin (α-Lac), Her2/neu, BRCA-2 or BRCA-1 (RNF53), KNG1K438-R457 (kininogen-1 peptide) and C3fS1304-R1320 (peptides that distinguish BRCA1 mutated from other BC and non-cancer mutated BRCA1).

In still another embodiment, the cancer-associated antigen is a colorectal cancer associated disease antigen including but not limited to MUCI, KRAS, CEA (CAP-1-6-D [Asp6]; YLSGADLNL—SEQ ID NO: 14) and AdpgkR304M MC38 (MHCI-Adpgk: ASMTNMELM SEQ ID NO: 15; MHCII-Adpgk: GIPVHLELASMTNMELMSSIVHQQVFPT SEQ ID NO: 16).

In still another embodiment, the cancer-associated antigen is a pancreatic cancer associated disease antigen including but not limited to CEA, CA 19-9, MUCI, KRAS, p53mut (peptide antigen of mouse mutated p53_{R172H} sequence VVRHCPHHER—SEQ ID NO: 4 (human mutated p53_{R175H} sequence EVVRHCPHHE—SEQ ID NO: 5)) and MUC4 or MUC13, MUC3A or CEACAM5, KRAS peptides (e.g. KRAS-G12R, KRAS-G13D, p5-21 sequence KLVVVGAGGVGKSALTI (SEQ ID NO: 17), p5-21 G12D sequence KLVVVGADGVGKSALTI (SEQ ID NO: 18), p17-31 sequence SALTIQLIQNHFVDE (SEQ ID NO: 19), p78-92 sequence FLCVFAINNTKSFED (SEQ ID NO: 20), p156-170 sequence FYTLVREIRKHKEKM (SEQ ID NO: 21), NRAS (e.g. NRAS-Q61R), PI3K (e.g. PIK3CA-H1047R), C-Kit-D816V, and BRCA mutated epitopes YIHTHTFYV (SEQ ID NO: 22) and SQIWNLNPV (SEQ ID NO: 23) HLA-A*02:01 restricted neoepitopes.

In still another embodiment, the cancer-associated antigen is a lung cancer associated disease antigen including but not limited to Sperm Protein 17 (SP17), A-kinase anchor protein 4 (AKAP4) and Pituitary Tumor Transforming Gene 1 (PTTG1), Aurora kinase A, HER2/neu, and p53mut.

In still another embodiment, the cancer-associated antigen is a prostate cancer associated disease antigen such as prostate cancer antigen (PCA), prostate-specific antigen (PSA) or prostate-specific membrane antigen (PSMA).

In another embodiment, the cancer-associated antigen is a heterologous protein or peptide to the pathogenic bacteria and the host cells but is native to a cancer tumor.

In another embodiment, the cancer-associated antigen is a neoantigen.

As used herein the term “neoantigen” is an epitope that has at least one alteration that makes it distinct from the corresponding wild-type, parental antigen, e.g., via mutation in a tumor cell or post-translational modification specific to a tumor cell. A neoantigen can include a polypeptide sequence or a nucleotide sequence. A mutation can include a frameshift or non-frameshift deletion, missense or nonsense substitution, splice site alteration, genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen.

An example of a mutant APC antigen is QATEAERSF (SEQ ID NO: 3).

Examples of BRCA mutated epitopes are YIHTHTFYV (SEQ ID NO: 22) and SQIWNLNPV (SEQ ID NO: 23) HLA-A*02:01 restricted neoepitopes.

An examples of a universal HLA-DR-binding T helper synthetic epitope (AKFVAAWTLKAAA, SEQ ID NO: 24) is the pan DR-biding epitope (PADRE), which is a 13 amino acid peptide that activates CD4+ T cells.

Another contemplated cancer-associated neoantigen is the GL261 neoantigen (mlmp3 D81N, sequence AALLNKLYA—SEQ ID NO: 6).

The bacteria described herein include a polynucleotide encoding the cancer-associated antigen operably linked to transcriptional regulatory elements, such as a bacterial promotor. In some embodiments, the bacterial promoter is inducible, endogenous or constitutive. In some embodiments, the bacterial promoter is endogenous. In some embodiments, the bacterial promoter is constitutive. In some embodiments, the bacterial promoter is inducible. In another embodiment, the polynucleotide is chromosomally integrated.

The bacteria described herein are genetically modified to express the cancer associated antigen, intracellularly and/or on the bacterial surface (i.e., genetic surface display). In another embodiment, the bacteria are genetically modified to secrete the cancer associated antigen. In another embodiment, the bacteria are genetically modified to express the cancer associated antigen constitutively.

For example, in some embodiments, the bacteria comprises a nucleic acid encoding the cancer-associated antigen operably linked to transcriptional regulatory elements, such as a bacterial promotor. In some embodiments, the bacteria comprise a polynucleotide encoding the cancer-associated antigen operably linked to a nucleic acid sequence which encodes signal peptide of a transport system.

The terms signal peptide” and “leader sequence” may be used interchangeably herein and refer to an amino acid sequence that can be linked at terminus of a protein or peptide set forth herein. Signal peptides/leader sequences typically direct localization of a protein such that it is secreted from, or positioned on, the outer membrane of the bacteria by facilitating surface display of the protein or peptide on the outer wall of the bacteria. The signal peptides/leader sequences used herein may facilitate secretion of the protein from the cell in which it is produced. The secretion signal may be categorized as belonging to a transport system selected from: Type II or Type III which refers to the specialized protein system, which directs the export of a protein, or alternatively, a wall presenting system. Type II secretion systems may alternatively be referred to as two-step secretion system, while the Type III secretion system may alternatively be referred to as a one-step secretion, typically secreted directly into a host cell. Signal peptides/leader sequences may be cleaved from the remainder of the protein, often referred to as the mature protein, upon secretion from the cell however, in some cases, there is no protease cleavage site between the transport signal and the neoantigen. Signal peptides/leader sequences may be linked at the amino terminus (i.e., N terminus) of the protein. Typically, the recombinant host cell engineered to express the fusion peptide or protein linked to the secretion signal is a bacterium having a functional secretion system of that signal.

As used herein, a signal sequence is an amino acid sequence or nucleotide sequence depending on context, which encodes the signal peptide.

It will be readily understood by those skilled in the art and it is intended here, that when reference is made to particular signal sequence listings, such reference includes sequences which substantially correspond to its complementary sequence and those described including allowances for minor sequencing errors, single base changes, deletions, substitutions and the like, such that any such sequence variation corresponds to the nucleic acid sequence of the signal peptide or other peptide/protein to which the relevant sequence listing relates.

Secretion signals of bacterial type III secretion systems are known by those skilled in the art and may include the Ssph1, Ssph2, MISSSSIS, MISSSSSI, sicP-sptP, sigE-sopB, invB-sopA, SptP, SipA, SipB, SipC, SipD, InvJ, SpaO, AvrA, and SopE proteins of Salmonella, the YopE, YopH, YopM and YpkA proteins of Yersinia spp., the Ipa proteins of Shigella, and the ExoS proteins of Pseudomonas aeruginosa and have been engineered as fusion proteins and peptides by many investigators^{a,b,c,d}. In some embodiments, the secretion signals of bacterial type III secretion systems is selected from sspH2, sspH1, sigE-sopB and sipB. For example, of nucleotide sequences encoding signal sequences include those of the type 3 secretion system (e.g. MISSSIS (SEQ ID 25) sequence NO: (DNA sequence: ATGATCAGCTCTAGTTCAATCAGC—SEQ ID NO: 26 or the MISSSSSI (SEQ ID NO: 27) sequence (DNA sequence: ATGATCAGCTCTAGTTCAAGCATC—SEQ ID NO: 28).

Secretion signals of those of the Type II secretion system (secreted out of the bacteria) e.g., PelB sequence (DNA sequence: ATGAAATACCTGTTGCCTACGGCAGCCGCTGGATTGTTATTACTCGCGGCCCAACCG GCCATGGCC—SEQ ID NO: 29) are known by those skilled in the art. Some examples include pelB, ompA, PSP (SEC family), yebF.

According to another aspect, the vaccine includes a pharmaceutically acceptable carrier and Gram-negative bacteria genetically modified to express (e.g. co-express) at least two cancer-associated antigens, wherein a first of the at least two cancer-associated antigens is linked to a first signal peptide and a second of the at least two cancer-associated antigens is linked to a second signal peptide, the first signal sequence belonging to a type II secretion system and the second signal sequence belonging to a type III secretion system. Furthermore, the same cancer associated antigen may be expressed and individually linked to two different signal sequences in the same bacteria.

In some embodiments, the cancer-associated antigen (e.g., neoantigen) is constitutively expressed by the bacteria (e.g., is under expression of a constitutive promoter). Examples of contemplated constitutive promoters include, but are not limited to PagC, Ssph2, sicA, pLac, J23105, J23119, J23105 promoters.

In some embodiments, the cancer-associated antigen is inducibly expressed by the bacteria (e.g., it is expressed upon exposure to aspirin, a sugar or an environmental stimulus like low pH or an anaerobic environment). In some embodiments, the bacteria comprises a plurality of nucleic acid sequences that encode for multiple different cancer-associated antigens that can be expressed by the same bacterial cell.

In some embodiments, the bacteria displays a recombinantly produced cancer-associated antigen on its surface using a bacterial surface display system. Examples of bacterial surface display systems include outer membrane protein systems (e.g., LamB, FhuA, Ompl, OmpA, OmpC, OmpT, eCPX derived from OmpX, OprF, and PgsA), surface appendage systems (e.g., F pillin, FimH, FimA, FliC, and FliD), lipoprotein systems (e.g., INP, Lpp-OmpA, PAL, Tat-dependent, and TraT), and virulence factor-based systems (e.g., AIDA-1, EacA, EstA, EspP, MSP1a, and invasin). Exemplary surface display systems are described, for example, in van Bloois, E., et al., Trends in Biotechnology, 2011, 29:79-86, which is hereby incorporated by reference. For example, the bacteria may be engineered to display the RGD peptide sequence (ACDCRGDCFCG—SEQ ID NO: 30) on the external loop of outer membrane protein A (OmpA).

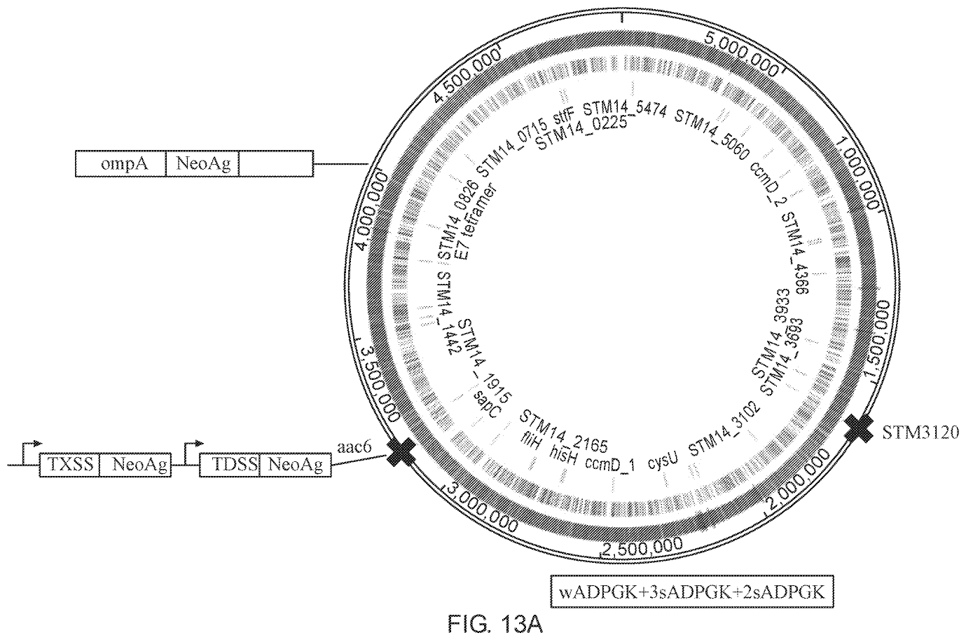

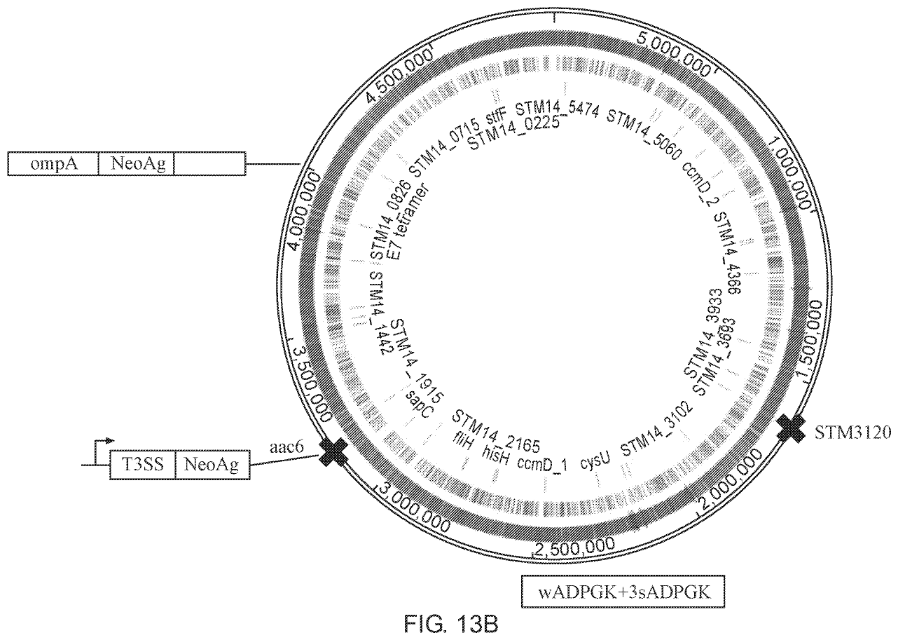

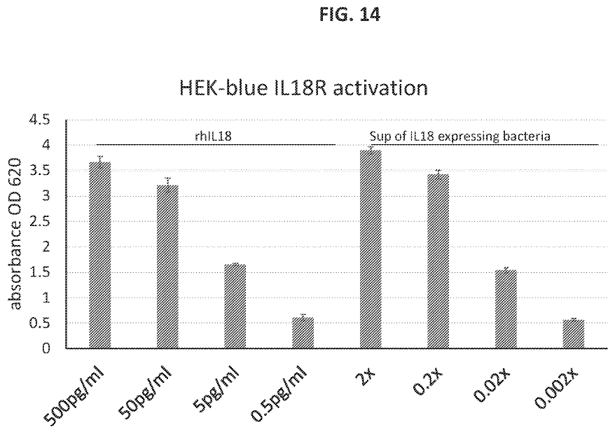

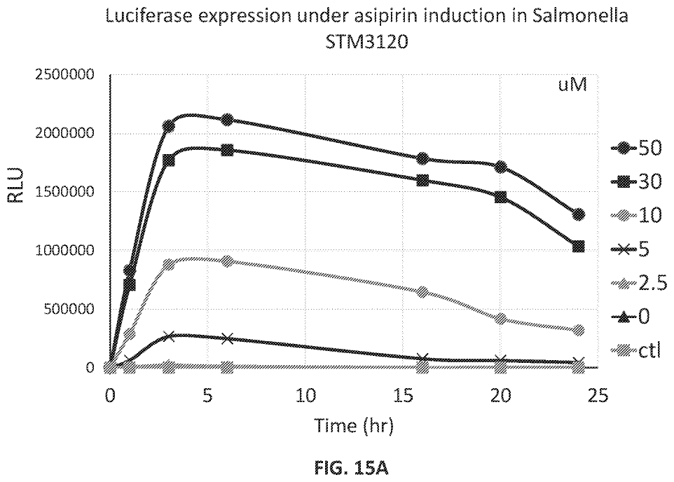

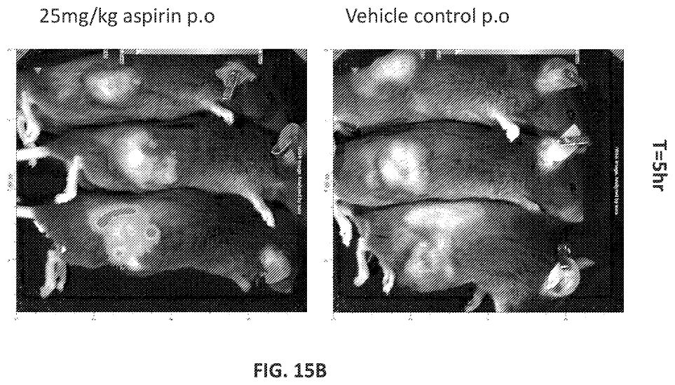

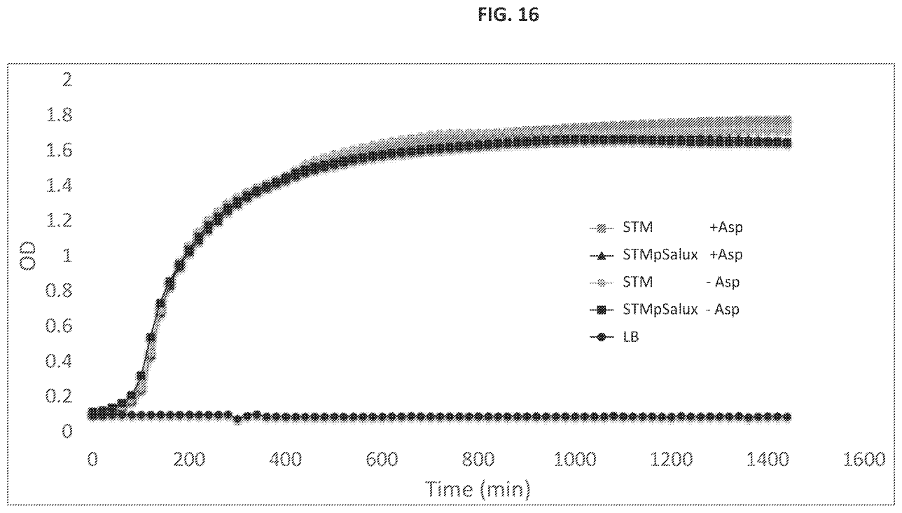

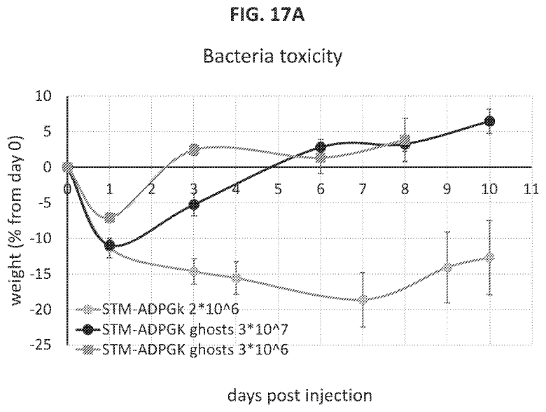

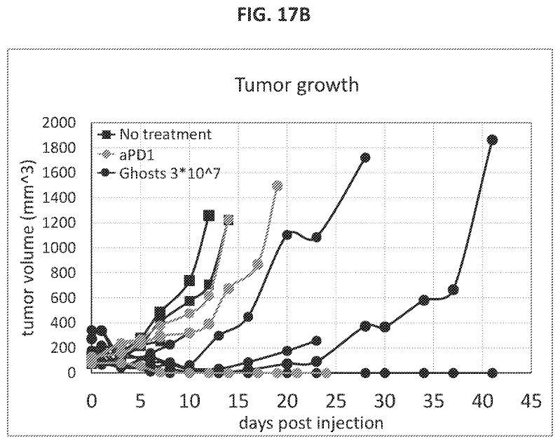

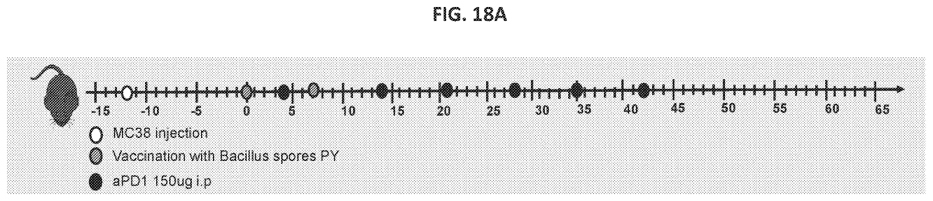

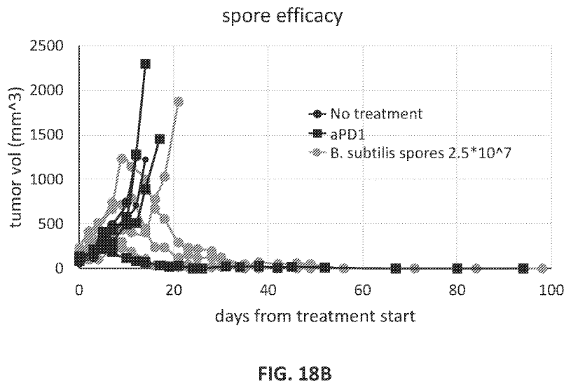

According to another aspect, bacteria are genetically modified to express (e.g. co-express) at least two cancer associated antigens, wherein a first of the at least two cancer-associated antigens comprises a first signal sequence and a second of the at least two cancer-associated antigens comprises a second signal sequence, the first signal sequence belonging to a type II or III secretion system (such that the cancer-associated antigen is secreted out of the bacteria) and the second signal sequence allows the cancer-associated antigen to be expressed on the outer membrane protein of the protein (e.g. by incorporating it into an ompA protein, as described herein above)—see for example FIG. 13. Furthermore, the same cancer-associated antigen may be expressed using two different signal sequences in the same bacteria—one for secretion outside the bacteria and one for presentation on the outer bacterial cell wall. In some embodiments, the cancer-associated antigen is a neoantigen.

In another aspect, bacteria are genetically modified to express (e.g., co-express) a cancer-associated antigen linked to a type II secretion system peptide and the same cancer-associated antigen linked to a type III secretion system peptide in the same bacteria (e.g., Gram negative bacteria). Such bacteria may further express the same cancer-associated antigen linked to an outer bacterial cell wall targeting moiety. In some embodiments, the cancer-associated antigen is a neoantigen.

In another aspect, a recombinant tumor colonization pathogenic gram-negative bacterium (e.g., Salmonella typhimurium) includes two or more chromosomally integrated prokaryotic expression cassettes. Typically, the prokaryotic expression cassettes are in frame.

In some embodiments, the recombinant tumor colonization pathogenic gram-negative bacterium (e.g., Salmonella typhimurium) includes prokaryotic expression cassettes encoding two copies of the same neoantigen or a set of neoantigens. In some embodiments, the recombinant tumor colonization pathogenic gram-negative bacterium (e.g., Salmonella typhimurium) includes prokaryotic expression cassettes encoding three copies of a neoantigen or a set of neoantigens. In some embodiments, each copy is associated with a signal sequence from a distinct transport system selected from Type II, III or wall.

As used herein, prokaryotic expression cassettes are configured to be expressed within a prokaryotic cell. Typically, the DNA construct of the prokaryotic expression cassette is operably linked to a prokaryotic promoter or further includes a prokaryotic promoter. In some examples, the promoter is a constitutive or endogenous promoter. Each of the two or more expression cassettes include a polynucleotide sequence which encodes a neoantigen or a series thereof, linked to a transport signal sequence from a distinct transport system selected from Type II, Type III or wall. For example, the first polynucleotide sequence may encode a neoantigen or a first series linked to a transport signal sequence from a first transport system, while the second polynucleotide sequence may encode the neoantigen or the series thereof, linked to a transport signal sequence from a second transport system, wherein the first and second are distinct and selected from Type II, Type III or wall. In another example, the first polynucleotide sequence may encode a neoantigen or a neoantigen series linked to a transport signal sequence from a first transport system, the second polynucleotide sequence may encode the neoantigen or the neoantigen series thereof, linked to a transport signal sequence from a second transport system, and a third polynucleotide sequence may encode a neoantigen or a neoantigen series linked to a transport signal sequence from a third transport system,

Typically, the pathogenic, gram-negative bacteria engineered for tumor colonization include two or more transport signals selected from the list consisting of: wall, type II, or type III.

The neoantigen series may be two or more, three or more, or four or more neoantigens in a series. The neoantigen series may include between 1 and 15, 1 and 10, 1 and 4 or 1 and 3 neoantigens in a series. In some embodiments, the neoantigen or series thereof of the first and second polynucleotide cassettes are the same.

The neoantigen cassette may include nucleotides encoding additional amino acids which do not adversely affect the secretory function of the signal peptide or do not adversely affect the function of the heterologous protein. For example, additional amino acids may be included which separate the signal peptide from the heterologous cancer associated antigen in order to provide a favored steric configuration in the fusion peptide which promotes the secretion process.

In some embodiments, DNA construct are organized sequentially with promoter, secretion signal, protein/peptide.

Examples of bacterial promoters include but are not limited to STM1787 promoter, pepT promoter, pflE promoter, ansB promoter, vhb promoter, FF+20* promoter or p(luxI) promoter.

According to a particular embodiment the promoter is an ompC promoter (SEQ ID NO: 31), a salRpSal promoter (SEQ ID NO: 32) or a J23109 promoter (SEQ ID NO: 33).

In some embodiments, the genetically modified bacteria described herein comprise a cancer therapeutic (e.g., the cancer therapeutic is loaded into the bacteria prior to administration to a subject or is genetically modified to express the cancer therapeutic).

In some embodiments, the cancer therapeutic is loaded into the bacteria by growing the bacteria in a medium that contains a high concentration (e.g., at least 1 mM) of the cancer therapeutic, which leads to either uptake of the cancer therapeutic during cell growth or binding of the cancer therapeutic to the outside of the bacteria. The cancer therapeutic can be taken up passively (e.g., by diffusion and/or partitioning into the lipophilic cell membrane) or actively through membrane channels or transporters. In some embodiments, drug loading is improved by adding additional substances to the growth medium that either increase uptake of the molecule of interest (e.g., Pluronic F-127) or prevent extrusion of the molecules after uptake by the bacterium (e.g., efflux pump inhibitors like Verapamil, Reserpine, Carsonic acid, or Piperine). In some embodiments, the bacteria is loaded with the cancer therapeutic by mixing the bacteria with the cancer therapeutic and then subjecting the mixture to electroporation, for example, as described in Sustarsic M., et al., Cell Biol., 2014, 142 (1): 113-24, which is hereby incorporated by reference. In some embodiments, the cells can also be treated with an efflux pump inhibitor (see above) after the electroporation to prevent extrusion of the loaded molecules.

In still further embodiments, the bacteria is genetically modified to express the cancer therapeutic. The bacteria may be genetically modified to co-express the cancer therapeutic (e.g. immunomodulatory) and the cancer associated antigen.

In some embodiments the bacteria of the vaccine comprise an inhibitory antibody or small molecule directed against the immune checkpoint protein—e.g. anti-CTLA4, anti-CD40, anti-41BB, anti-OX40, anti-PD1 and anti-PDL1.

The present inventors further contemplate that the bacteria of the vaccine may comprise therapeutic agents attached to the outside of the bacteria using an attachment method such as CLICK chemistry. Such methods are further described in U.S. patent application No. 20200087703 and U.S. patent application No. 20200054739, the contents of which are incorporated herein by reference.

Examples of therapeutic agents include immune modulatory proteins (i.e., immunomodulators), such as a cytokine. Examples of immune modulating proteins include, but are not limited to, B lymphocyte chemoattractant (“BLC”), C—C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interlukin-1 alpha (“IL-1 alpha”), Interlukin-1 beta (“IL-1 beta”), Interleukin 1 receptor antagonist (“IL-1ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C—C motif) Ligand 2 (“MCP-1”), Macrophage colony-stimulating factor (“M-CSF”), Monokine induced by gamma interferon (“MIG”), Chemokine (C—C motif) ligand 2 (“MIP-1 alpha”), Chemokine (C—C motif) ligand 4 (“MIP-1 beta”), Macrophage inflammatory protein-1-delta (“MIP-1 delta”), Platelet-derived growth factor subunit B (“PDGF-BB”), Chemokine (C—C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (′BMP-5″), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”). Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing complex (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C—C motif) ligand 27 (“CTACK”), Chemokine (C—X—C motif) ligand 16 (“CXCL16”), C—X—C motif chemokine 5 (“ENA-78”), Chemokine (C—C motif) ligand 26 (“Eotaxin-3”), Granulocyte chemotactic protein 2 (“GCP-2”), GRO, Chemokine (C—C motif) ligand 14 (“HCC-1”), Chemokine (C—C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C—X—C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C—C motif) ligand 20 (“MIP-3 alpha”), C—C motif chemokine 19 (“MIP-3 beta”), Chemokine (C—C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C—C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”). Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R. IL-2Rgamma, IL-21R. Lysosome membrane protein 2 (“LIMPII”), Neutrophil gelatinase-associated lipocalin (“Lipocalin-2”), CD62L (“L-Selectin”), Lymphatic endothelium (“LYVE-1”). MEC class I polypeptide-related sequence A (“MICA”), MEC class I polypeptide-related sequence B (“MICB”), NRG1-beta1, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A. Agouti-related protein (“AgRP”), Ribonuclease 5 (“Angiogenin”). Angiopoietin 1. Angiostatin, Catheprin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C. FoUistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgp130, Secreted frizzled-related protein 2 (“ShhN”), Sialic acid-binding immunoglobulin-type lectins (“Siglec-5”), ST2, Transforming growth factor-beta 2 (“TGF beta 2”), Tic-2. Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), CAMP receptor protein (“CRP”), Human Epidermal Growth Factor Receptor 2 (“ErbB2”), Follistatin, Follicle-stimulating hormone (“FSH”), Chemokine (C—X—C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21. Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C—X—C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST. Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor .kappa. B (“RANK”).

According to a specific embodiment, the immune modulator is one of the following proteins:

Interleukin-18 (IL-18) (e.g. SEQ ID NOs: 34-37), Tumor Necrosis Factor Superfamily Member 14 (LIGHT) (e.g. SEQ ID NOs: 38-39), CD40 Ligand (CD40L) (e.g. SEQ ID NO: 40-43), Signal Regulatory Protein Alpha (SIRPa) (e.g. SEQ ID NO: 44)C—C Motif Chemokine Ligand 5 (CCL5) (e.g. SEQ ID NO: 45), Anti-IL10R1 peptide (e.g. SEQ ID NO: 46), Granulocyte-macrophage colony stimulating factor (GM-CSF) (e.g. SEQ ID NO: 47), C—C Motif Chemokine Ligand 21 (CCL21) (e.g. SEQ ID NO: 48-49), Short Salmonella flagellin B (fliC) (e.g. SEQ ID NO: 50) and DacA (e.g. SEQ ID NO: 51).

The immune modulatory protein can be made recombinantly using methods known to one skilled in the art. The immune modulatory protein can be presented on the surface of a bacterium using bacterial surface display, where the bacterium expresses a genetically engineered protein-protein fusion of e.g., a membrane protein and the immune modulatory protein.

The bacteria of the vaccine of the present invention may serve as an adjuvant, thereby rendering the use of additional adjuvant not relevant.

In one embodiment, the vaccine is devoid of adjuvant (other than the bacteria itself).

In another embodiment, the vaccine comprises an adjuvant additional to the bacteria.

Adjuvants are substance that can be added to an immunogen or to a vaccine formulation to enhance the immune-stimulating properties of the immunogenic moiety. Examples of adjuvants or agents that may add to the effectiveness of proteinaceous immunogens include aluminum hydroxide, aluminum phosphate, aluminum potassium sulfate (alum), beryllium sulfate, silica, kaolin, carbon, water-in-oil emulsions, and oil-in-water emulsions. A particular type of adjuvant is muramyl dipeptide (MDP) and various MDP derivatives and formulations, e.g., N-acetyl-D-glucosaminyl-(.beta.1-4)-N-acetylmuramyl-L-alanyl-D-isoglutami-ne (GMDP) (Hornung, R L et al. Ther Immunol 1995 2:7-14) or ISAF-1 (5% squalene, 2.5% pluronic L121, 0.2% Tween 80 in phosphate-buffered solution with 0.4 mg of threonyl-muramyl dipeptide; see Kwak, L W et al. (1992) N. Engl. J. Med., 327:1209-1238). Other useful adjuvants are, or are based on, cholera toxin, bacterial endotoxin, lipid X, whole organisms or subcellular fractions of the bacteria Propionobacterium acnes or Bordetella pertussis, polyribonucleotides, sodium alginate, lanolin, lysolecithin, vitamin A, saponin and saponin derivatives such as QS21 (White, A. C. et al. (1991) Adv. Exp. Med. Biol., 303:207-210) which is now in use in the clinic (Helling, F et al. (1995) Cancer Res., 55:2783-2788; Davis, T A et al. (1997) Blood, 90:509), levamisole, DEAE-dextran, blocked copolymers or other synthetic adjuvants. A number of adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.), Amphigen (oil-in-water), Alhydrogel (aluminum hydroxide), or a mixture of Amphigen and Alhydrogel. Aluminum is approved for human use.

As mentioned, the vaccines described herein may be used to treat and/or prevent cancer.

As used herein, the term “treating” includes abrogating, substantially inhibiting, slowing or reversing the progression of a condition, substantially ameliorating clinical or aesthetical symptoms of a condition.

According to a particular embodiment, the term preventing refers to substantially preventing the appearance of clinical or aesthetical symptoms of a condition.

Particular subjects which are treated are mammalian subjects—e.g., humans.

According to a particular embodiment, the subject has been diagnosed as having cancer.

### Cancer

The term “cancer” as used herein refers to an uncontrolled, abnormal growth of a host's own cells which may lead to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s).” “neoplasm(s),” and “tumor(s)” are used herein interchangeably. As used herein, “cancer” refers to all types of cancer or neoplasm or malignant tumors including leukemias, carcinomas and sarcomas, whether new or recurring.

Specific examples of cancers that may be treated using the bacteria described herein include, but are not limited to adrenocortical carcinoma, hereditary; bladder cancer; breast cancer; breast cancer, ductal; breast cancer, invasive intraductal; breast cancer, sporadic; breast cancer, susceptibility to; breast cancer, type 4; breast cancer, type 4; breast cancer-1; breast cancer-3; breast-ovarian cancer; triple negative breast cancer, Burkitt's lymphoma; cervical carcinoma; colorectal adenoma; colorectal cancer; colorectal cancer, hereditary nonpolyposis, type 1; colorectal cancer, hereditary nonpolyposis, type 2; colorectal cancer, hereditary nonpolyposis, type 3; colorectal cancer, hereditary nonpolyposis, type 6; colorectal cancer, hereditary nonpolyposis, type 7; dermatofibrosarcoma protuberans; endometrial carcinoma; esophageal cancer; gastric cancer, fibrosarcoma, glioblastoma multiforme; glomus tumors, multiple; hepatoblastoma; hepatocellular cancer; hepatocellular carcinoma; leukemia, acute lymphoblastic; leukemia, acute myeloid; leukemia, acute myeloid, with eosinophilia; leukemia, acute nonlymphocytic; leukemia, chronic myeloid; Li-Fraumeni syndrome; liposarcoma, lung cancer; lung cancer, small cell; lymphoma, non-Hodgkin's; lynch cancer family syndrome II; male germ cell tumor; mast cell leukemia; medullary thyroid; medulloblastoma; melanoma, malignant melanoma, meningioma; multiple endocrine neoplasia; multiple myeloma, myeloid malignancy, predisposition to; myxosarcoma, neuroblastoma; osteosarcoma; osteocarcinoma, ovarian cancer; ovarian cancer, serous; ovarian carcinoma; ovarian sex cord tumors; pancreatic cancer; pancreatic endocrine tumors; paraganglioma, familial nonchromaffin; pilomatricoma; pituitary tumor, invasive; prostate adenocarcinoma; prostate cancer; renal cell carcinoma, papillary, familial and sporadic; retinoblastoma; rhabdoid predisposition syndrome, familial; rhabdoid tumors; rhabdomyosarcoma; small-cell cancer of lung; soft tissue sarcoma, squamous cell carcinoma, basal cell carcinoma, head and neck; T-cell acute lymphoblastic leukemia; Turcot syndrome with glioblastoma; tylosis with esophageal cancer; uterine cervix carcinoma, Wilms' tumor, type 2; and Wilms' tumor, type 1, and the like.

According to a particular embodiment, the cancer is cancer is selected from the group consisting of breast, melanoma, pancreatic cancer, ovarian cancer, bone cancer and brain cancer (e.g., glioblastoma).

According to another embodiment, the cancer is melanoma.

Malignant melanomas are clinically recognized based on the ABCD (E) system, where A stands for asymmetry, B for border irregularity, C for color variation, D for diameter>5 mm, and E for evolving. Further, an excision biopsy can be performed in order to corroborate a diagnosis using microscopic evaluation. Infiltrative malignant melanoma is traditionally divided into four principal histopathological subgroups: superficial spreading melanoma (SSM), nodular malignant melanoma (NMM), lentigo maligna melanoma (LMM), and acral lentiginous melanoma (ALM). Other rare types also exists, such as desmoplastic malignant melanoma. A substantial subset of malignant melanomas appear to arise from melanocytic nevi and features of dysplastic nevi are often found in the vicinity of infiltrative melanomas. Melanoma is thought to arise through stages of progression from normal melanocytes or nevus cells through a dysplastic nevus stage and further to an in-situ stage before becoming invasive. Some of the subtypes evolve through different phases of tumor progression, which are called radial growth phase (RGP) and vertical growth phase (VGP).

In a particular embodiment, the melanoma is resistant to treatment with inhibitors of BRAF and/or MEK.

The tumor may be a primary tumor or a secondary tumor (i.e. metastasized tumor).

The compositions may be administered using any route such as for example oral administration, rectal administration, topical administration, inhalation (nasal) or injection. Administration by injection includes intravenous (IV), intramuscular (IM), intratumoral (IT), subtumoral (ST), peritumoral (PT), and subcutaneous (SC) administration. The pharmaceutical compositions described herein can be administered in any form by any effective route, including but not limited to intratumoral, oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), intravesical, intrapulmonary, intraduodenal, intragastrical, and intrabronchial. In some embodiments, the pharmaceutical compositions described herein are administered orally, rectally, intratumorally, topically, intravesically, by injection into or adjacent to a draining lymph node, intravenously, by inhalation or aerosol, or subcutaneously. In some embodiments, the pharmaceutical compositions described herein configured for administration by intravenous (IV), intramuscular (IM), intratumoral (IT), subtumoral (ST), peritumoral (PT), and subcutaneous (SC) administration. In some embodiments, the bacteria are administered intratumorally. In some embodiments, the bacteria are administered by injection. In certain embodiments, the bacteria are administered to the site of the tumor, intratumorally or intravenously. In a particularly preferred embodiments, the pharmaceutical compositions described herein are administered intravenously for systemic exposure.

According to another aspect of the present invention there is provided a method of treating cancer of a subject in need thereof comprising administering to the subject a therapeutically effective amount of:
(i) a first vaccine comprising a first bacteria which is genetically modified to express at least one cancer-associated antigen; and subsequently
(ii) a second vaccine comprising a second bacteria which is genetically modified to express at least one cancer-associated antigen, thereby treating the cancer.

The present invention contemplates at least two different vaccination cycles for the treatment of cancer, wherein at least one of the vaccination cycles includes one strain of genetically modified bacteria and at least another of the vaccination cycles includes a second (non-identical) genetically modified strain of bacteria. The two strains of bacteria may be genetically modified to express the same cancer associated antigens or different cancer associated antigens. Additionally, or alternatively, the present inventors contemplate at least one of the vaccination cycles includes viable bacteria (e.g., the first vaccination) and at least another of the vaccination cycles (e.g., a subsequent vaccination) includes non-viable bacteria—for example bacterial ghosts or vice versa.

Alternatively, the two different vaccines (one comprising viable, attenuated bacteria and the other comprising non-viable bacteria, such as a bacterial ghost) may be co-administered.

The vaccine of the present invention may be administered with additional anti-cancer agents.

In some embodiments the additional anti-cancer agent is an inhibitory antibody or small molecule directed against the immune checkpoint protein—e.g. anti-CTLA4, anti-CD40, anti-41BB, anti-OX40, anti-PD1 and anti-PDL1.

Other contemplated anti-cancer agents which may be administered to the subject in combination with the bacteria described herein include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Materials and Methods

### Plasmids and DNA Fragments for Genomic Insertions:

To generate backbone plasmids for Salmonella typhimurium strains, Ssph2 promoter and secretion signal (aa: 1-200), or the pagC promoter and Ssph1 secretion signal (aa: 1-208) were amplified from the Salmonella typhimurium attenuated strain VNP20009. Ssph2 and pagC-Ssph1 were inserted into pQE60 by NEBbuilder cloning kit (cat. E5520S).

To generate DNA fragments with homologous arms for the genomic integration of neoantigens, amplified fragments (promoter, secretion signal, neoantigen and homologous arms) were assembled into a linear fragment using NEBbuilder, followed by PCR amplification of the assembled fragment.

Proteins of interest were fused with either Ssph1 or Ssph2. To generate a backbone plasmid for Pseudomonas aeruginosa, proteins of interest were fused with the N-terminal 54 amino acids of ExoS in plasmid pEAI3-S54 (a courtesy of Bertrand Toussaint, PMID: 17010670). To generate a backbone plasmid for Bacillus subtilis spores, proteins of interest were fused with CotC (amplified from Bacillus subtilis 168) and cloned into pDG364 plasmid. In addition, 6His tag element was inserted to allow detection of the protein product.

### Neoantigens:

To obtain a neoantigen of B16-OVA tumors, the C-terminal of Ovalbumin (aa 252-386) was amplified from pcDNA-OVA (Addgene 64599). The amplified oligo contains the sequence which corresponds to SIINFEKL (SEQ ID NO: 11), the epitope of Ovalbumin.

To obtain a neoantigen of MC38 tumors, a section of Adpgk (aa 289-421) was amplified from cDNA of MC38 cells. The amplified oligo contains a sequence which corresponds to a validated neoantigen of MC38, based on Yadav et al. (PMID: 25428506).

Both neoantigens were inserted to the backbone plasmids by NEBuilder cloning kit, when episomal expression is tested, or integrated into the bacterial genome using the two-step scar-less lambda red recombineering method.

### Bacteria:

The attenuated Salmonella typhimurium strains VNP20009 (also named YS1646, ATCC, cat. 202165) and STM3120 were transformed with the relevant plasmids by electroporation. Briefly, bacteria were cultured to OD of 0.6-0.8, washed 3 times with Hepes 1 mM and suspended in 10% glycerol in DDW. Suspension was electroporated with 0.2 cm, cuivette (BioRad, EC2) and moved to 1 ml cold SOC. Following 1 hour incubation in 37° C., bacteria were seeded on LB agar plate containing ampicilin. Selected colonies were verified by Sanger sequencing and Western blot using anti-6His tag (Cell Signalling, 2365S).

The attenuated Pseudomonas aeruginosa (CHA-OST) was transformed as described by Diver et al. PMID: 2126169. The Bacillus subtilis strain PY79 was transformed following incubation in minimal medium and 0.01M MGSO₄ in DDW (MC: 80 mM K₂HPO₄, 30 mM KH₂PO₄, 2% Glucose, 30 mM Trisodium citrate, 22 μg/ml Ferric ammonium citrate, 0.1% Casein Hydrolysate (CAA), 0.2% potassium glutamate) for 3 hours to induce competent bacteria. Next, plasmid pDG364 which contains an antigen fused to CotC protein was cut with Xba and incubated with competent bacteria for 3 hours. Upon integration into the amylase gene, colonies were selected by resistance to chloramphenicol 5 μg/ml.

In all transformations, selected colonies were verified by Sanger sequencing and Western blot using anti-6His tag (Cell Signalling, 2365S).

Freezing Working Stocks of Salmonella typhimurium:

Exponentially growing culture (OD 0.6-0.8) was washed twice in cold PBS. Bacteria pellet was suspended in 25% glycerol in PBS. A sample from the bacterial stock was serially diluted and seeded on LB agar plate, while the rest of the pool was aliquoted and stored in −80° C. To verify viability of bacteria, a frozen aliquot was defrosted and seeded on LB agar plate. Recovery rate following freezing was quantified by calculating the ratio of frozen/fresh CFU count. Calculation of bacteria dosage in mice experiment was based on the CFU count of the frozen culture.

Sporulation of Bacillus subtilis PY79:

PY79 were grown in LB, at 37° C. to OD 0.8. LB medium was removed and replaced by half volume of DSM exhaustion medium. Culture was incubated at 37° C., whilst shaking for 60 hrs. Finally, bacteria were washed twice in cold water. To quantify spores, and sporulation rate, a sample from the washed sample was seeded on LB agar plate pre- and post 1 hour heating at 65° C. The ration of heated/non heated CFU count is indicative of sporulation rate. Exhaustion medium preparation (per 1 liter): dissolve 8 g Difco nutrient broth (BD, cat. 234000), 1 g KCl and 1 mM MgSO₄ in DDW. Titrate with NaOH to PH7.6 and autoclave. Before usage, add 10 μM MnCl₂, 1 mM Ca (NO₃)₂ and 1 mM FeSO₄.

### Mice Models:

B16-OVA mouse melanoma cell line (10⁶) or MC38 mouse CRC cell line (10⁵) were injected s.c. to the right flank of 7 weeks C57BL/6 females. Tumor volume was calculated as width\hat{ }2*length/2.

### Immune Profiling of Splenocytes by FACS:

Freshly resected spleens were mashed on a 70-micron strainer into cold PBS. To lyse red blood cells, the splenocytes were incubated with ACK lysis buffer (Quality Biological, cat. 118-156-101), then washed thoroughly in PBS and suspended in FACS labeling buffer. 100 μl of splenocytes were incubated for 1 hour at 4° C. with a mixture containing Fc blocker (BD, cat. 553142, 1:100), SIINFEKL (SEQ ID NO: 11) Tetramer (NIH Tetramer Core Facility, 1:500), anti-CD4 (BioLegend, cat. 100438, 1:800), anti-CD8 (Invitrogen, cat. 2021 May 5, 1:400), anti CD3 (Invitrogen, cat. 2023 Jul. 31, 1:1000) and Brilliant Buffer (BD, cat. 566349, 1:5). Next, cells were washed twice in labeling buffer and fixed with CytoFix/CytoPerm solution (BD, cat. 51-2090KZ) for 20 mins at 4° C. Finally, cells were washed twice in Perm/Wash buffer (BD, cat. 51-2091KZ, diluted in DDW 1:10) suspended in labeling buffer and subjected to FACS.

### Quantification of Activated CD8 T Cells by Peptide Stimulation

Splenocytes were produced as described above. Next, splenocytes were incubated with OVA peptide (final conc. 2.5 μg/ml) for 2 hours at 37° C. Next, Brafeldin A (BD, 51-2301kz) was added to the cells and incubated for additional 4 hours at 4° C. FACS staining for CD3, CD8 and INFg were performed the next day as described above.

### Ex Vivo Killing Assay.

MC38 or B16-OVA cells were seeded on 48 well plate. Cells were stained with CFSE (5 uM) for 20 min at 37° C., then quenched with culture medium (RPMI with 10% FCS) for 10 min at 37° C. and washed twice with culture medium.

The next day, spleens were resected as described above and cells were counted. Next, 10⁵ splenocytes were co-cultured with the tumor cells and incubated for 72 hours at 37° C.

Following incubation, FACS staining for DEAD/LIVE (Invitrogen, L34962) and CFSE positive (tumor cells) were performed the next day as described.

### IFNg Quantification by ELISA:

To quantify serum level of IFNg, mice were bled into Eppendorf tube containing 20 μl Heparin (10 mg/ml). Following centrifugation for 10 mins, 10,000 g, sera were transferred to new tubes for long term storage at −20° C. ELISA was performed according to manufacturer instructions (R&D, cat. DY485) using sera diluted 1:4.

### Bacteria Quantification in Liver and Tumor:

Slices of tumors and livers were suspended in sterile tubes containing LB and metal beads. Following vortex for 10 minutes at max speed, 200 μl of sup, were seeded on LB plates with the relevant antibiotics and incubated over night at 37° C.

### Results

To demonstrate the efficacy of the Personalized Anti-Cancer Microbiome-Assisted Vaccination (PACMAN) vaccine, bacteria expressing the Ovalbumin known neoantigen SIINFEKL (SEQ ID NO: 11) were administered to mice bearing the B16 melanoma tumors which express the Ovalbumin protein (B16-OVA). To generate the OVA expressing bacteria vaccine, the OVA neoantigen SIINFEKL (SEQ ID NO: 11) was fused to Ssph2 secretion signal of Salmonella typhimurium. The resulted oligomer was transformed into the attenuated Salmonella typhimurium strain VNP20009 (VNP-OVA). C57BL/6 mice were injected with 10⁶ B16 OVA expressing cells in the right flank. When tumors reached a volume of ˜100 mm³, mice were shuffled into the following treatment cohorts: No treatment control, mice receiving the checkpoint inhibitor, anti-PD1 (75 μg per mouse, i.p, once a week), and mice receiving anti-PD1 together with PACMAN-OVA (10⁶ CFU, tail vein). The experiment timeline is shown in FIG. 1A. Tumor growth curves from treatment start are shown in FIG. 1B. Tumor growth was completely stopped for 20 days in the PACMAN-OVA cohort versus the exponential growth observed in the other mice cohorts. Following two cycles of immunization, all mice in the VNP-OVA cohort survived significantly longer than the mice in the other cohorts.

To demonstrate the immunogenicity of the vaccine, splenocytes were profiled from mice bearing the B16-OVA tumor following the administration of the PACMAN vaccine. The PACMAN-OVA contained the OVA neoantigen SIINFEKL (SEQ ID NO: 11) fused to Ssph2 secretion signal of Salmonella typhimurium in the attenuated strain STM3120. For a negative control the OVA neoantigen was replaced by the MC38 neoantigen, Adpgk (PACMAN-Adpgk), which is not present in B16-OVA cells.

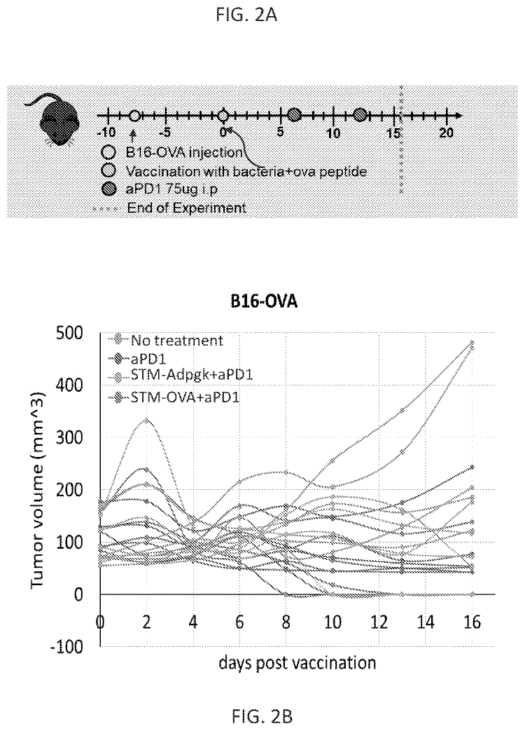

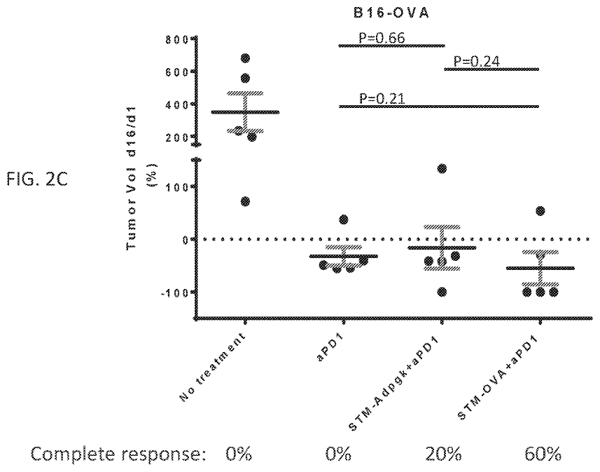

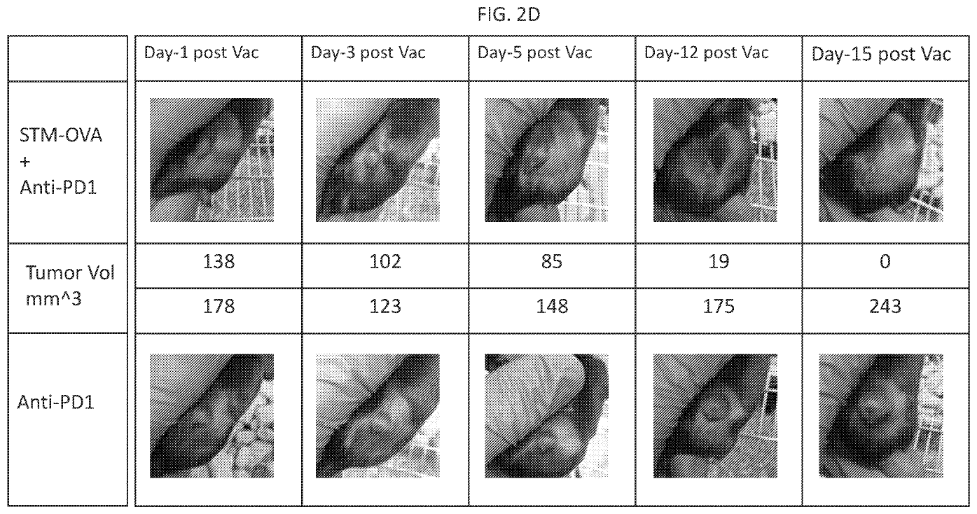

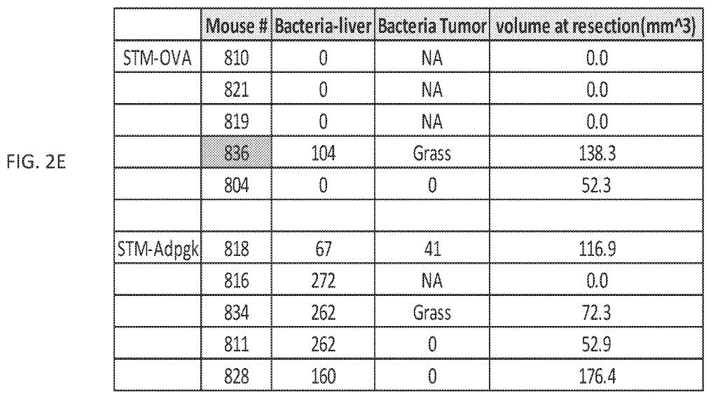

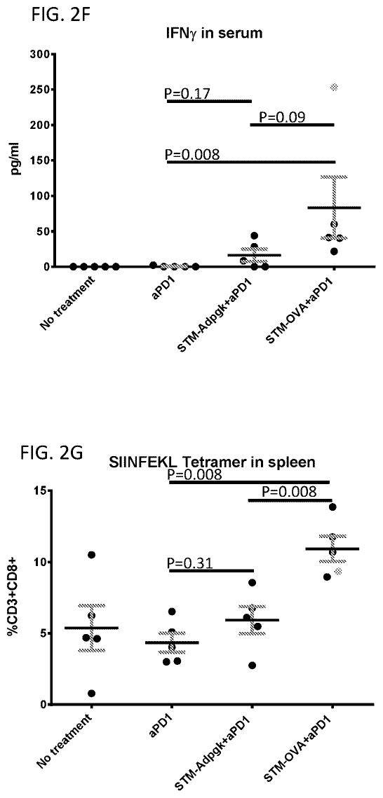

C57BL/6 mice were injected with 10⁶ B16 OVA expressing cells in the right flank. When tumors reached a volume of ˜100 mm³, mice were shuffled into the following treatment cohorts: No treatment control, mice receiving the checkpoint inhibitor, anti-PD1 (75 μg per mouse, i.p, once a week), mice receiving anti-PD1 together with PACMAN-OVA (10⁶ CFU, tail vein) and mice receiving anti-PD1 with PACMAN-Adpgk (10⁶ CFU, tail vein). Experiment setup and efficacy of the treatment (tumor volume) are illustrated in FIGS. 2A-D. Blood was taken from mice at day 16 and serum levels of IFNγ were measured with ELISA (FIG. 2F). Sixteen days post immunization, tumors, spleens and liver were harvested for the evaluation of bacterial load (CFU/gr/ml, FIG. 2E) and quantification of neoantigen specific T cell clones, measured with a SIINFEKL (SEQ ID NO: 11)-tetramer assay on harvested splenocytes (FIG. 2G).

To test the effect of alternate administration of PACMAN-OVA, which is based on different attenuated bacteria, mice bearing B16-Ova tumor were vaccinated consecutively with two attenuated bacteria expressing the OVA neoantigen. The first bacteria is the Salmonella attenuated strain STM3120 expressing Ova neoantigen fused to either SshpH2 secretion signal under its endogenous promoter or to Ssph1 secretion signal under pagC promoter which is induced upon phagocytosis by macrophages (STM-OVA). The second bacteria is the Pseudomonas aeruginosa attenuated strain, CHA-OST, expressing Ova neoantigen fused to the secretion signal of ExoS, a toxin of the type-three secretion system (TTSS). ExoS promoter is activated by the TTSS regulator ExsA, following induction by IPTG (CHA-OST-OVA).

C57BL/6 mice were injected with 10⁶ B16 OVA expressing cells in the right flank. When tumors reached a volume of ˜100 mm³, mice were shuffled into the following treatment cohorts: No treatment control, mice receiving the checkpoint inhibitor, anti-PD1 (75 μg per mouse, i.p, once a week), mice receiving anti-PD1 together with STM-SspH2-OVA and mice receiving anti-PD1 together with STM-pagC-SspH1-OVA. The vaccinated mice were treated with 3 doses of STM-OVA (10⁶ CFU, tail vein), followed by anti-PD1 (75 μg per mouse, i.p, once a week). Two weeks since the last STM-OVA vaccine, the mice were treated with 2 doses of CHA-OST-OVA (10⁷ CFU, tail vein, following 3 hours incubation with IPTG 0.5 mM). As illustrated in FIG. 3B, tumor growth was significantly delayed in the mice which were vaccinated with STM-OVA compared to non-vaccinated mice. The majority of tumors in the vaccinated mice regained growth 20-30 days post vaccination, suggesting that the additional injections of the same bacteria did not contribute enough to anti-tumor immunity. Strikingly, vaccinating the mice with CHA-OST-OVA slowed down tumor growth and, in some cases, even caused exponential decay. As illustrated in FIG. 3C and FIG. 3D, weight decrease is observed following each bacteria administration, however, weight loss is less pronounced after additional vaccination with the same bacteria, further supporting the hypothesis that the mice develop immunity towards the bacteria resulting in fast clearance and thus less effect on body weight.

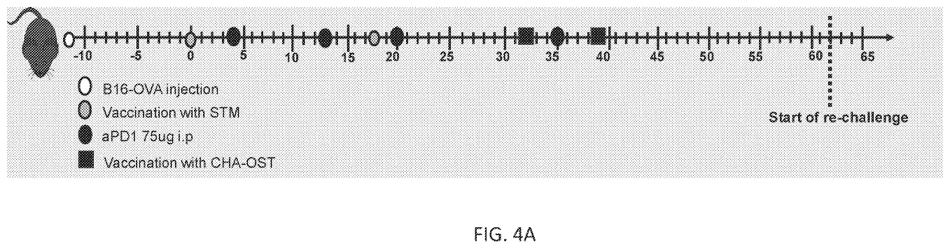

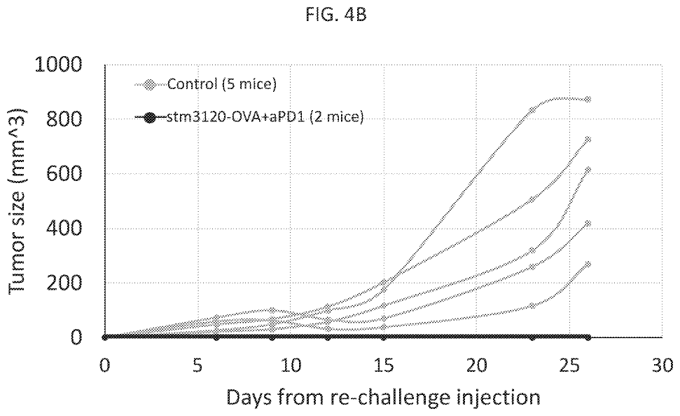

To test the immune memory of mice vaccinated with PACMAN-OVA, fully cured mice from the experiment described in FIG. 3A were re-challenged with 10⁶ B16-Ova cells and tumor growth was compared to naïve mice injected with the same number of cells. As illustrated in FIG. 4B, based on experimental timeline in 4A, while naïve mice exhibited exponentially growing tumors shortly after injection, re-challenged mice remained tumor free, indicating the establishment of long-term immune memory against B16-Ova cells.

To demonstrate the efficacy of the PACMAN vaccine with naturally occurring neoantigen in another mouse model, the effect of bacteria expressing the Adpgk neoantigen of MC38 model was tested on mice bearing the MC38 CRC tumors (FIG. 5). To generate the Adpgk expressing bacteria vaccine, the Adpgk neoantigen was fused to Ssph1 secretion signal of Salmonella Typhimurium under pagC promoter which is induced upon phagocytosis by macrophages. Next, the oligomer was transformed into the attenuated Salmonella typhimurium strain VNP20009 (PACMAN-Adpgk). C57BL/6 mice were injected with 10⁵ MC38 cells in the right flank. When tumors reached a volume of ˜100 mm³, mice were shuffled into the following treatment cohorts: mice receiving the checkpoint inhibitor, anti-PD1 (75 μg per mouse, i.p, once a week), mice receiving anti-PD1 together with VNP20009 and mice receiving anti-PD1 together with PACMAN-Adpgk (10⁶ CFU, tail vein) according to the treatment timeline in FIG. 5A. Tumor growth curves from treatment start are presented in FIG. 5B. As shown, tumor growth was significantly inhibited in the PACMAN-Adpgk cohort relative to the other mice cohorts. Following two cycles of immunization, one mouse in the PACMAN-Adpgk cohort was fully cured.

To test the immune memory of mice vaccinated with PACMAN-Adpgk, the mice exhibiting full cure following vaccination with PACKMAN-Adpgk or VNP20009 (w/o adpgk) were re-challenged with 10⁵ MC38 cells and tumor growth was compared to naïve mice injected with the same number of cells. While naïve mice exhibited exponentially growing tumors shortly after injection, re-challenged mouse which was vaccinated with PACKMAN-Adpgk remained tumor free, indicating the establishment of long-term immune memory against MC38 cells. Of note, the fully cured mouse following vaccination only with the VNP20009, exhibited tumor growth following re-challenge indicating that the immune memory was a consequence of Adpgk presentation by the bacteria (FIG. 5C).

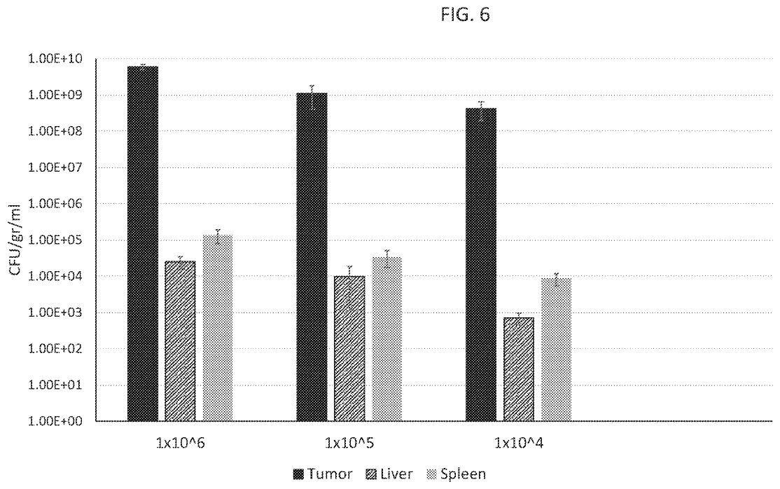

To demonstrate selective colonization of Salmonella to MC38 tumors, attenuated Salmonella (STM3120) was injected to the tail vein of mice bearing the MC38 CRC tumors at the indicated numbers. After 9 days, tumors, livers and spleens were resected and vigorously shaken in 1 ml LB and a metal ball. Supernatant was seeded on LB plates and colonies were counted following 24 hrs incubation at 37° C. CFU was normalized to the dilution factor and tissue mass. For 1×10⁶, 1×10⁵ N=4, for 1×10⁴ N=3. As illustrated in FIG. 6, the bacteria selectively homed to the tumors as compared to livers and spleens.

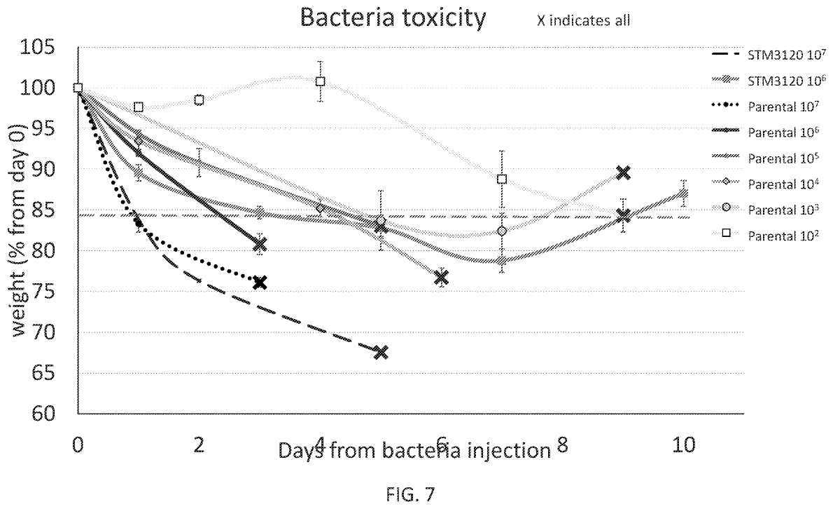

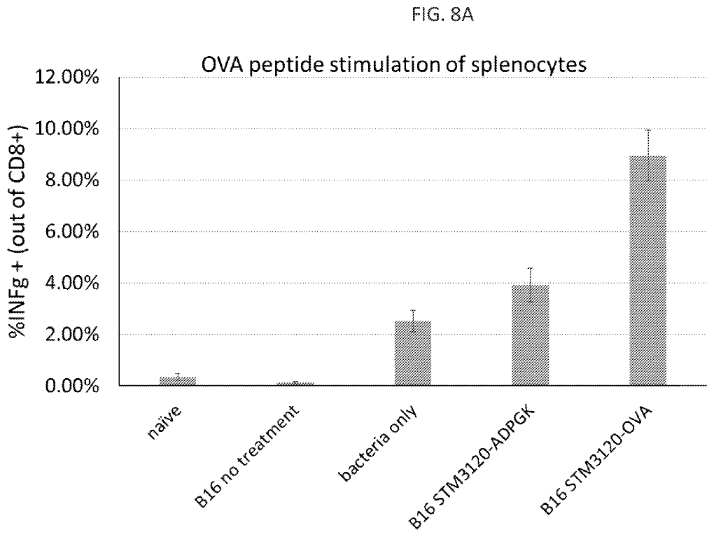

To compare the maximal tolerable dose of attenuated Salmonella (STM3120) vs parental Salmonella (14028), Salmonella were injected to the tail vein at various concentrations and body weight was monitored. As illustrated in FIG. 7, in all doses but STM3120 1e6, all mice in the cohort (N=4-5) died (indicated by X).

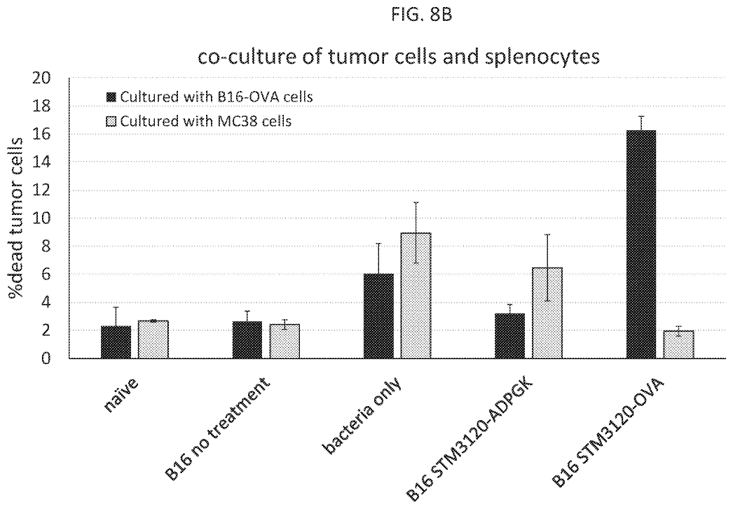

In order to quantify the amount of active T cells following PACMAN vaccination, splenocytes were harvested from the following cohorts: naïve mice (N=3), B16-OVA tumor bearing mice (N=5), mice injected with attenuated Salmonella STM3120 (N=4), B16 OVA tumor bearing mice injected with STM3120 expressing the unrelated neoantigen ADPGK (N=3), B16-OVA tumor bearing mice injected with STM3120 expressing the OVA neoantigen (N=5). In all cases, 1e6 bacteria were injected to the tail vein. Sixteen days post injection, splenocytes were harvested. FIG. 8A illustrates the increase in IFNg positive CD8 T-cells following vaccination with the appropriate neoantigen.

In a further experiment to quantify T cell killing capacity, MC38 or B16-OVA tumor cells were pre-incubated with CFSE (green) to distinguish them from immune cells. Harvested splenocytes were co-cultured with tumor cells. Following 72 hours, dead tumor cells (CFSE positive) were quantified by FACS using Live/dead staining. Significant B16-OVA specific killing was observed in splenocytes originating from mice vaccinated with STM3120 expressing the OVA neoantigen (Two-tail t-test, Pval<0.001).

To demonstrate the immune mediated efficacy of P. aeruginosa based PACMAN vaccine in MC38 colorectal cancer model, the attenuated P. aeruginosa, CHA-OST either naïve or expressing Adpgk neoantigen was injected to the tail vein, followed by anti PD1 treatment. C57BL/6 mice were injected with 1×10⁵ MC38 colorectal cancer cells in the right flank. When tumors reached a volume of ˜100 mm³, mice were injected with CHA-OST naïve or PACMAN-ADPGK (1×10⁶ CFU, i.v) followed by weekly administration of 150 μg anti-PD1, i.p. FIG. 9A is a graphic representation of the treatment protocol. As illustrated in FIGS. 9B and 9C, only mice which were injected with the PACMAN-ADPGK vaccine showed a full cure. Of note, the cured mouse was re-challenged with MC38 cells, however no tumor growth was observed.

To demonstrate the immune mediated efficacy of Bacillus subtilis based PACMAN vaccine in MC38 colorectal cancer model, the spores of the lab strain PY79 expressing Adpgk neoantigen were injected to the tail vein or administered orally (os), followed by aPD1 treatment. C57BL/6 mice were injected with 5×10⁵ MC38 colorectal cancer cells in the right flank. When tumors reached a volume of ˜100 mm³, mice were injected with bacillus spores of PACMAN-ADPGK (5×10⁸-1×10⁹ CFU, i.v) or given per os (5×10⁹ CFU, p.o) followed by weekly administration of 150 μg anti-PD1, i.p. FIG. 10A is a graphic representation of the treatment protocol. As illustrated in FIG. 10B, mice which were injected with the PACMAN-ADPGK vaccine showed a full cure.

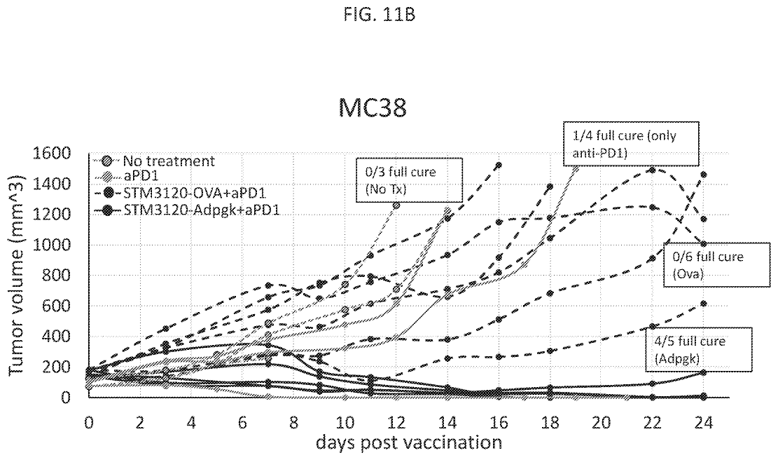

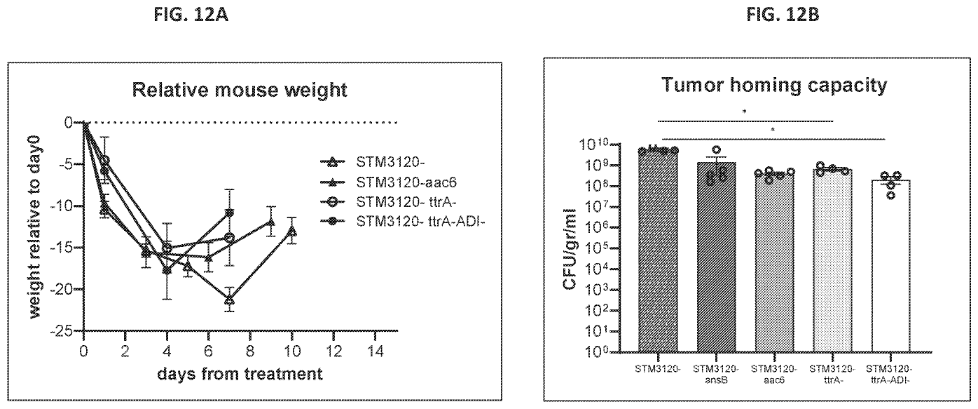

To demonstrate the immune mediated efficacy of Salmonella based PACMAN vaccine (which has previously been frozen) in MC38 colorectal cancer model, the attenuated Salmonella Typhimurium STM3120 expressing related (Adpgk) and unrelated (OVA) neoantigen was injected to the tail vein followed by aPD1 treatment. C57BL/6 mice were injected with 10⁶ B16 OVA expressing cells in the right flank. When tumors reached a volume of ˜ 100 mm³, mice were injected with PACMAN-ADPGK or PACMAN-OVA (3×10⁶ CFU, i.v) followed by weekly administration of 75/150 μg anti-PD1, i.p. FIG. 11A is a graphic representation of the treatment protocol. As illustrated in FIG. 11B, mice treated with PACMAN-ADPGK exhibited a considerable delayed tumor growth.

### Example 2

Attenuated strains: Knock-out strains were generated using the two-step scar-less lambda red recombineering method. As a first step, lambda red recombineering was used to introduce a cassette that expresses the TetA and SacB genes. This insertion rendered the bacteria sensitive to sucrose (sacB gene) and resistant to tetracycline (TetA gene) and can be accordingly selected. In the second step, tetA-sacB cassette was replaced with a sequence of interest rendering the bacteria sensitive to tetracyline and resistant to sucrose (loss of sacB gene). The bacterial strains with successful integration can accordingly be counter-selected using sucrose. This method enables the bacterial genome to be engineered with insertions or knockouts without leaving any unwanted DNA sequences (“scars”) in the bacterial genome.

Three knockout strains (STM3120-aac6-, STM3120-ttraA- and STM3120-ttraA-adI-) were generated and their toxicity and colonizing capacity was evaluated and compared to STM3120- strain. Reduction in bacterial toxicity was observed (measured by body weight loss) together with minor reduction in tumor colonization (FIGS. 12A-B).

STM3120 were genetically modified to express IL-18. Secretion and functional activity was tested. As illustrated in FIG. 16, a substantial activation of the target receptor following the addition of the bacterial supernatant, indicating that the bacteria secrete the functional payload.

### Inducible Expression of Payloads in Bacteria:

As illustrated in FIGS. 15A-B, expression of luciferase was induced under control of the pSal promoter and the salR aspirin biosensor in Salmonella STM3120. Exponentially growing STM3120 expressing luciferase under induction of Aspirin were incubated with different concentration of Aspirin. Luminescence was quantified in duplicates over 24 hours. RLU-relative luminescence units.

FIG. 15B shows Aspirin-induction of expression of luciferase in Salmonella STM3120 in mice. Mice bearing MC38 sub-cutaneous tumors (>100 mm\hat{ }3) were injected I.V with STM3120 expressing luciferase, 10\hat{ }6 CFU per mouse. Twenty-four hours post injection, mice were gavaged with either vehicle control or Aspirin 25 mg/kg and luminescence was read 5 hours later. Color bar represents relative luminescence unit. range: 0-1×10\hat{ }6 RLU.

FIG. 16 illustrates that Aspirin does not affect growth of Salmonella typhimurium (STM3120). STM3120 (STM) bacteria and STM3120 bacteria harboring a plasmid with aspirin inducible luciferase expression (STM pSal-lux) were grown with 200 uM aspirin (+Asp) or without Aspirin (−Asp) and OD was measured to reflect their growth rate. LB-Luria Broth medium with no bacteria.

Generation of bacterial ghosts: Bacteria were grown to OD 0.6-0.8. Next, bacteria were washed twice with PBS following centrifugation at 4000 g, for 10 minutes. Then, bacteria pellet was resuspended in 4% PFA solution and incubated overnight in shaking at 37° C. The fixated bacteria were washed twice in PBS. While in PBS, CFU/ml was estimated based on O.D read. Finally, the resulting bacteria ghosts were resuspended in 50% glycerol in PBS and stored in −80° C.

In vivo experiments (FIGS. 17A-B) demonstrate a strong anti-tumor effect following the injection of neo-antigen expressing STM3120 ghost bacteria. It was also found that injection of ghost bacteria leads to a much lower weight loss and a much faster recovery from initial weight loss as compared to live bacteria.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

### BIBLIOGRAPHY

¹ Winter K, Xing L, Kassardjian A, Ward B J. Vaccination against Clostridium difficile by Use of an Attenuated Salmonella enterica Serovar Typhimurium Vector (YS1646) Protects Mice from Lethal Challenge. Infect Immun. 2019 Jul. 23; 87 (8): e00089-19. doi: 10.1128/IAI.00089-19. PMID: 31138615; PMCID: PMC6652760.
¹ Yoon W, Park Y C, Kim J, Chae Y S, Byeon J H, Min S H, Park S, Yoo Y, Park Y K, Kim B M. Application of genetically engineered Salmonella typhimurium for interferon-gamma-induced therapy against melanoma. Eur J Cancer. 2017 January; 70:48-61. doi: 10.1016/j.ejca.2016.10.010. Epub 2016 Nov. 22. PMID: 27883926.
¹ Yoon W, Choi J H, Kim S, Park Y K. Engineered Salmonella typhimurium expressing E7 fusion protein, derived from human papillomavirus, inhibits tumor growth in cervical tumor-bearing mice. Biotechnol Lett. 2014 February; 36 (2): 349-56. doi: 10.1007/s10529-013-1370-8. Epub 2013 Oct. 22. PMID: 24146199.
¹ Widmaier D M, Tullman-Ercek D, Mirsky E A, Hill R, Govindarajan S, Minshull J, Voigt C A. Engineering the Salmonella type III secretion system to export spider silk monomers. Mol Syst Biol. 2009; 5:309. doi: 10.1038/msb.2009.62. Epub 2009 Sep. 15. PMID: 19756048; PMCID: PMC2758716.

What is claimed is:

1. A vaccine comprising a pharmaceutically acceptable carrier and Gram-negative bacteria, genetically modified to express:
at least one disease-associated antigen, linked to a signal sequence belonging to a type II secretion system; and
said at least one disease-associated antigen linked to a signal sequence belonging to a type III secretion system.

2. A vaccine comprising a ghost bacteria of the species Salmonella enterica, said bacteria being genetically modified to express at least one disease-associated antigen on a cell wall of said bacteria.

3. The vaccine of claim 1 or 2, wherein said bacteria are genetically modified to further express:
at least one disease-associated antigen linked to a bacterial outer membrane targeting sequence.

4. The vaccine of claim 1, wherein said Gram-negative bacteria comprise a bacterium which is genetically modified to co-express:
said at least one disease-associated antigen, linked to said signal sequence belonging to a type II secretion system; and
said at least one disease-associated antigen linked to said signal sequence belonging to a type III secretion system.

5. The vaccine of claim 1 or 4, wherein said Gram negative bacteria are of the species Salmonella enterica.

6. The vaccine of any one of claims 1-5, wherein said at least one disease-associated antigen is a cancer-associated antigen.

7. The vaccine of claim 6, wherein said cancer-associated antigen is expressed in said bacteria under control of a constitutive promoter.

8. The vaccine of claim 6 or 7, wherein said cancer-associated antigen is a neoantigen.

9. The vaccine of any one of claims 1-8, wherein said bacteria are genetically modified to express a reduced amount or an inactive product of a gene selected from the group consisting of: STM3120, arginine deaminase (adI), L-asparaaginase II (asnB), Aminoglycoside (3″) (9) adenylyltransferase (aadA), AAC (6′)-Iaa (aac6) and Tetrathionate reductase A (ttrA).

10. The vaccine of claim 9, wherein said bacteria are genetically modified to express a reduced amount or an inactive product of three genes selected from the group consisting of STM3120, arginine deaminase (adI), L-asparaaginase II (asnB), Aminoglycoside (3″) (9) adenylyltransferase (aadA), AAC (6′)-Iaa (aac6) and Tetrathionate reductase A (ttrA).

11. The vaccine of claim 2 or 5, wherein said bacteria are of a serotype typhimurium.

12. The vaccine of claim 9, wherein at least one of said three genes is STM3120.

13. The vaccine of any one of claims 1-12, wherein said bacteria are capable of homing to a tumor of a subject having cancer following i.v. administration.

14. An attenuated bacteria of the species Salmonella enterica genetically modified to express a reduced amount or a less active product of at least one gene selected from the group consisting of arginine deaminase (adI), L-asparaaginase II (asnB), Aminoglycoside (3″) (9) adenylyltransferase (aadA), AAC (6′)-Iaa (aac6) and Tetrathionate reductase A (ttrA) as compared to non-attenuated bacteria of the species Salmonella enterica.

15. The attenuated bacteria of claim 14, further comprising a mutation in STM3120.

16. The attenuated bacteria of claim 14, being capable of homing to a tumor of a subject having cancer following i.v. administration.

17. A vaccine comprising the attenuated bacteria of claim 14 or 16, genetically modified to express at least one disease-associated antigen and a pharmaceutically acceptable carrier.

18. The vaccine of claim 17, wherein said bacteria are genetically modified to express at least one cancer-associated antigen.

19. The vaccine of claim 18, wherein said at least one cancer-associated antigen comprises a signal sequence.

20. The bacteria of claim 14 or 16, or vaccine of any one of claims 17-19, wherein said bacteria are genetically modified to express at least two cancer-associated antigens, wherein a first of said at least two cancer-associated antigens comprises a first signal sequence and a second of said at least two cancer-associated antigens comprises a second signal sequence, said first signal sequence belonging to a type II secretion system and said second signal sequence belonging to a type III secretion system.

21. The bacteria or vaccine of claim 20, wherein said bacteria are genetically modified to express at least three cancer-associated antigens, wherein a first of said at least three cancer-associated antigens comprises a first signal sequence, a second of said at least three cancer-associated antigens comprises a second signal sequence, and a third of said at least three cancer-associated antigens comprises a sequence for embedding into an outer wall of said bacteria, said first signal sequence belonging to a type II secretion system and said second signal sequence belonging to a type III secretion system.

22. The bacteria or vaccine of any one of claims 1-21, wherein said bacteria are genetically modified to express an immunomodulator.

23. The bacteria or vaccine of claim 22, wherein said immunomodulator is selected from the group consisting of Interleukin-18 (IL-18), Tumor Necrosis Factor Superfamily Member 14 (LIGHT), Signal Regulatory Protein Alpha (SIRPa), CD40 Ligand (CD40L), C—C Motif Chemokine Ligand 5 (CCL5), Anti-IL10R1 peptide, Granulocyte-macrophage colony stimulating factor (GM-CSF), C—C Motif Chemokine Ligand 21 (CCL21), Short Salmonella flagellin B (fliC) and DacA.

24. The bacteria or vaccine of claim 22 or 23, wherein said immunomodulator is expressed in said bacteria under control of an inducible promoter.

25. The bacteria or vaccine of claim 24, wherein said inducible promoter is an aspirin inducible promoter.

26. A method of treating cancer of a subject in need thereof comprising administering to the subject a therapeutically effective amount of the vaccine or bacteria of any one of claims 1-25, thereby treating the cancer.

27. The method of claim 26, wherein said administering is by intravenous (i.v.) injection.

28. The method of claim 26 or 27, wherein the vaccine is the vaccine of claim 2 and the method further comprises administering to the subject:
a second vaccine comprising a viable bacteria genetically modified to express at least one cancer-associated antigen, thereby treating the cancer.

29. The method of claim 28, wherein said second vaccine is administered following said vaccine of claim 2.

30. The method of claim 28, wherein said second vaccine is administered concomitantly with said vaccine of claim 2.

31. The method of any one of claims 26-30, wherein the cancer is selected from the group consisting of breast, melanoma, colorectal cancer, lung cancer, gastric cancer, pancreatic cancer, ovarian cancer, bone cancer and brain cancer.

32. The method of claim 31, wherein said brain cancer comprises glioblastoma.

33. A method of preventing cancer of a subject in need thereof the method comprising administering to the subject a prophylactically effective amount of the vaccine or bacteria of any one of claims 1-25, thereby preventing the cancer.

34. The vaccine or bacteria of any one of claims 1-25, for use in treating cancer.

35. The method of claim 33, or vaccine for use of claim 34, wherein the cancer is selected from the group consisting of breast, melanoma, colorectal cancer, gastric cancer, lung cancer, pancreatic cancer, ovarian cancer, bone cancer and brain cancer.

36. The method or vaccine for use of claim 35, wherein said brain cancer comprises glioblastoma.
